SEQUENCE LISTING (11 sequences)

SEQ ID NO: 1 - protein, 671 aa, Yarrowia lipolytica
MTPHPFDQLSVQEMESVVRVVKSNHSGKSLHLKSIGTEEPPKALMAPFLAAKRAGKNPVPPPRIAHVIYYVLEDKLVNQCWVDVPSAKVVKSEVLKKGIHPPIDPWEANEAFEAAFDHPLVKDAIKKCGVEHLIDNLTIDGWMYGCDSEIDMPRYLQMLVYCRDPKTNHQDSNMYAFPVPFVPVYDVLEKKLVRVDYCATGGDDDDAAVEGVGNYDTRPEGKNCIEHCVTNDYLPELQDKMRTDLKPYNVLQPEGPSYHIDSDGYINWQKWHFKVGFTPREGLVIHDVHYDGRSTFYRLSMSEMAVPYADPRPPLHRKMAFDFGDCGGGKCANELTLGCDCLGTIRYFDGNVCDPEGNVFTRKNVICMHEQDDGIGWKHTNYRTDVVAITRRRILVLQTILTVGNYEYIFAWHFDQSAGIQLEIRATGIVSTQLIDAGKKSKFGTIVSPGVMAASHQHIFNVRMDPAIDGHQNTVVVNDTVALPWDAKNPHGIAFENTKTPIEKSCYLDSDIQKNRYLKICNENKINPISGNPVGYKIGGLATAMLYAQPGSVSRNRAAFATHHYWVTKYKDQELFAGGVWTNQSANEVGGVQDAVARNENVRNDDVVLWHSFGLTHHPRVEDFPVMPCEIMKIHLSPNDFFTGNPSVDVPKSNQTFNRSVEVKDCRSCKI

SEQ ID NO: 2 - DNA, 2016 bp, Yarrowia lipolytica
atgactccccaccctttcgatcagctctccgttcaggagatggagagcgttgtgcgagtggtcaagtccaaccattcgggcaagtctcttcacctcaagtccattggcaccgaggagcctcccaaggcgctgatggctcctttccttgcagccaagcgagctggcaagaaccccgttcccccccctcgaatcgcacatgtcatctactatgttctggaggacaagttggtgaaccagtgctgggtcgatgttccttccgccaaggttgtcaagtccgaggtgctcaagaagggcatccatcctcccattgatccctgggaggccaacgaggccttcgaggccgcctttgaccatcctctggtcaaggacgccatcaagaagtgcggcgtggagcatctgatcgacaacctcacaattgacggctggatgtacggctgtgacagcgagattgacatgccccggtacctgcagatgctggtctactgccgagatcccaagaccaaccaccaggactccaacatgtacgccttccccgttccgtttgttcctgtctacgacgtgctggagaagaagctcgttcgagtcgactattgcgccaccggtggagacgatgacgacgctgccgtcgagggcgttggcaactacgacacccgacccgagggaaagaactgcatcgagcactgtgtcaccaacgactatcttcccgagcttcaggacaagatgcgaaccgacctcaagccctacaacgtgttgcagcccgagggtccctcttaccacattgacagtgacggctacatcaactggcaaaagtggcacttcaaggtcggattcactccccgagagggtctggtgatccacgatgtccactacgacggccgatccaccttctaccgactgtccatgtccgagatggcggttccctacgccgatccccggccccctctgcaccgaaagatggcgtttgatttcggcgactgtggaggaggaaagtgcgccaacgagctgactctgggctgcgactgtcttggtaccatccgatactttgacggcaacgtgtgcgaccccgagggcaacgtgttcacccgaaagaacgtcatctgtatgcacgagcaggatgacggtatcggctggaagcacactaactaccgaaccgacgtggttgccatcacccgacgacgaattctggttctgcagaccattctgaccgtgggcaactacgagtacatctttgcctggcactttgaccagtctgccggaatccagctggagatccgagccaccggaatcgtctccacccagcttatcgacgccggcaagaagtccaagtttggcaccattgtctctcccggagtcatggccgcctctcaccagcacattttcaacgtgcgaatggaccctgccattgacggccatcagaacacagttgtggtcaacgacaccgttgctctgccctgggacgccaagaacccccatggaatcgcctttgagaacaccaagacccccatcgagaagtcgtgctacctggactcggacatccagaagaaccggtacctcaagatctgcaacgagaacaagatcaaccccatctccggcaaccccgtgggctacaagattggaggtctggccaccgccatgctgtacgctcagcccggctccgtttcgcgaaaccgagccgcctttgccacccaccactactgggtcaccaagtacaaggaccaggagctgtttgccggcggcgtgtggaccaaccagtctgccaacgaggtcggcggagtccaggatgccgttgcccgaaacgagaacgtgcgaaacgacgacgtggttctctggcactcctttggtctcactcaccacccccgagtcgaggacttccccgtcatgccttgcgagattatgaagatccatctgtcgcccaacgatttcttcaccggcaacccttctgttgacgtgcccaagtccaaccagactttcaatcgatccgtcgaggtcaaggactgtcgatcttgcaagatctag

SEQ ID NO: 3 - DNA, 2091 bp, Yarrowia lipolytica
atgcacagactatcacaactagctacacaaacaaccgcggccaccatcaccgcaggtcatcctcttgatcctctctctccctccgaaatcgaacatgccgcttccatcgtcaaatcgcagatgcgagactcgtctccgtaccggttcaatctcatcacgctgatcgagccgcccaaggccgagcttcttgcgtgggaggcgtcgccttcctcggtggccaaacctcctcgacgagcggaggtcgtactcgttgtgagaggcaagaagggcgtcaccgagggccgggtctgtctcaccggctcaaaggtgctttcgtggtccgaaattgagggcgtccagcctattcttaccgttgacgacctccaaaaggtcgaggaaattgtgcgacaggaccccgaagtcatcaagcagtgcaaactcattggagttgacaacatgtcccaggtgtactgtgatccctggactattggctatgacgaaagatggggtgccgaacgacgtctacagcaggcgttcctctacttccgagcccaccaggacgactcccagtactcccatcctcttgacttcactccaatctacgacgccacggagcagaaagtcatctttattgatattcctcccgttcgtcgacctctctccaagcttaagaattccaacttcaaccctcaggatatctccaagactaccggttacagagacgtgaagcccatcgacgtgtctcagcccgaaggagtcaacttcaagatgaccggtcgaatcatggagtggcagggattccgatttcacgtgggattcaactacagagagggaattgtgctgtctcagatctctttcaacgaccatggtaaccagcgaaacatgttccatcgtctctctctcgttgaaatggttgttccctacggaaaccccgagcaccctcaccagcgaaagcacgcctttgatctgggagagtacggagccggtctcatgaccaatcctctttccctcggatgtgattgcaagggagtcattcactaccttgatgcgcacttttccgacgccgaaggaaagcctctcactgttcccaacgctgtgtgtatccacgaggaggacaatggtctgcttttcaagcactctgacttccgagacgagttccagacttcgatcgtcactcgagctaccaagctcattttgtcgcagattttcaccgccgcaaactacgaatactgcgtctactggattttccaccaggacggtaccatccagctggagattaagctcaccggcatcctcaacactttcccctgcaatcccggagaagatttgcatggctggggcacagaggtgtaccctggtaccaacgcccacaaccatcagcatctcttttctctgcgaatccatcctgccattgattcccagctgcatccaggtaattctgtggcaatggtggacgctgagcggtcacccttcccgcctggacacccagaaaacctgcacggcaatggtttccggcccaagcgaacggtcttcaacaacccgatcgaggctatgacggattatgatggtaatacatcgcgaacttgggactttttcaacccgaagtccatcaaccagtactccaagaagcccgcttcttacaagctggtgtctcgtgagtgccctcctctgcttcctcagcctggtggactggtttggaaccgagctggttttgcacgacaccatatgcatgttgttccgtatgtggacggccagctgtaccctgctggacggtttgtttgccagacaagtggaaagccctcaaggggtctccccgagtggattgagcagtgcggagagaaggccaatatcaacgataccgatgttgttgcctatcacacttttggtctgacccatttccctgctcccgaggacttccctttgatgcctgccgagcccatgactctacttttgcgacccagaaacttcttcctgcagaacccggctttggacgtgcctccttcgcatgctcgaaccaccacagaggcccaggctgcttccggtgctaaggttgtctctcttaccgacaaggtgtcgcagctggctttcaataagtcgtgctgttccaagtag

SEQ ID NO: 4 - DNA, 24 bp, Artificial Sequence
  forward primer
atgactccccaccctttcgatcag

SEQ ID NO: 5 - DNA, 28 bp, Artificial Sequence
  Reverse primer
ctagatcttgcaagatcgacagtccttg

SEQ ID NO: 6 - DNA, 27 bp, Artificial Sequence
  Forward primer
cttgcgcgcatgactccccaccctttc

SEQ ID NO: 7 - DNA, 27 bp, Artificial Sequence
  Reverse primer
ggcgctagcctagatcttgcaagatcg

SEQ ID NO: 8 - DNA, 32 bp, Artificial Sequence
  Forward primer
gaagcgcgcatgcacagactatcacaactagc

SEQ ID NO: 9 - DNA, 27 bp, Artificial Sequence
  Reverse primer
caagctagcctacttggaacagcacga

SEQ ID NO: 10 - DNA, 25 bp, Artificial Sequence
  Forward sequencing primer
ctaaagatgttgatctccttgtgcc

SEQ ID NO: 11 - DNA, 21 bp, Artificial Sequence
  Reverse sequencing primer
cctctgggccgaatacaacac

(19) United States
(12) Patent Application Publication
(10) Pub. No.: US 2025/0320468 A1
(43) Pub. Date: Oct. 16, 2025

(54) New Microbial Diamine Oxidase Derived from Yarrowia Lipolytica for the Degradation of Biogenic Amines

(71) Applicant: UNIVERSITÄT HOHENHEIM, Stuttgart (DE)

(72) Inventors: Lutz Fischer, Notzingen (DE); Lucas Kettner, Stuttgart (DE); Ines Seitl, Ostfildern (DE); Sabine Lutz-Wahl, Kirchheim (DE)

(73) Assignee: UNIVERSITÄT HOHENHEIM, Stuttgart (DE)

(21) Appl. No.: 18/570,546

(22) PCT Filed: Mar. 22, 2022
(86) PCT No.: PCT/EP2022/057431

§ 371 Date: Dec. 14, 2023

(30) Foreign Application Priority Data
Jun. 15, 2021 (EU) EP21179434.2

(51) Int. Cl. C12N 9/06; A61K 9/00; A61K 9/20; A61K 38/00; A61P 37/08; C12N 15/80

(52) U.S. Cl. CPC C12N 9/0022; A61K 9/0053; A61K 9/2018; A61P 37/08; C12N 15/80; A61K 38/00; C12Y 104/03022

(57) ABSTRACT

The present invention relates to functional foods and dietary supplements comprising a specific diamine oxidase (DAO) enzyme derived from the yeast Yarrowia lipolytica PO1f, uses of said enzyme and respective methods for the production of biogenic amine-depleted products, and said enzyme for use in medicine, in particular for use in the prevention or treatment of a condition or disease that is associated with increased levels of biogenic amines.

### CROSS REFERENCE TO RELATED APPLICATIONS

This application is a 371 of PCT/EP2022/057431 filed Mar. 22, 2022, which claims priority to EP21179434.2 filed Jun. 15, 2021. The contents of each foregoing application are incorporated by reference in their entirety for all purposes.

### REFERENCE TO SEQUENCE LISTING, TABLE OR COMPUTER PROGRAM

The official copy of the Sequence Listing is submitted with the specification as an ASCII formatted text file, via EFS-Web, with a file name of “UHH002_ST25”, a creation date of Jun. 18, 2024, and a size of 13 kilobytes. The Sequence Listing filed via EFS-Web is part of the specification and is incorporated in its entirety by reference herein.

The present invention relates to functional foods and dietary supplements comprising a specific diamine oxidase (DAO) enzyme derived from the yeast Yarrowia lipolytica PO1f, uses of said enzyme and respective methods for the production of biogenic amine-depleted products, and said enzyme for use in medicine, in particular for use in the prevention or treatment of a condition or disease that is associated with increased levels of biogenic amines.

Biogenic amines such as histamine are especially found in foods that undergo a fermentation process due to the presence of microorganisms producing L-histidine decarboxylase. L-histidine decarboxylase (EC 4.1.1.22) generates histamine through the decarboxylation of the precursor L-histidine during the fermentation or storage of the food. Histamine exhibits a multitude of physiological functions in the human, acting as an important hormone and neurotransmitter. Therefore, the consumption of large amounts of exogenous histamine in food can cause serious poisoning with various physiological symptoms, such as vomiting, diarrhea or hypotension. Other biogenic amines, such as tyramine, putrescine or cadaverine, are also frequently found in foods and can also cause toxicological effects in the human body. The consumption of moderate or even small amounts of histamine can also cause adverse allergy-like reactions in some susceptible individuals. It is estimated that around 1% of the total population is intolerant towards histamine and, thereby, susceptible to minor dosages. This intolerance seems to derive from a disbalance between the amount of histamine ingested and the activity of the histamine degrading enzyme DAO (EC 1.4.3.22) available in the small intestine. This enzyme degrades histamine by oxidative deamination, resulting in the reaction products (imidazol-4-yl) acetaldehyde, hydrogen peroxide and ammonia.

There is currently no real treatment for the intolerance against biogenic amines such as histamine. People who are affected can administer a dietary supplement containing porcine DAO that is supposed to support the endogenous DAO in the small intestine. However, it has been shown that the activity required for a satisfactory histamine reduction is considerably larger than expected and that an alternative to the porcine DAO formulation used currently has to be found.

Specifically, DAO extracted from porcine kidney is commercially available in a dietary supplement and has already been investigated in clinical trials for its efficacy. However, it has recently been shown that this enzyme cannot be extracted and administered in sufficient quantities to achieve satisfactory histamine depletion. In particular, kinetic studies on porcine kidney DAO showed substrate inhibition by histamine concentrations greater than 56 mg/L (0.5 mM). The stability of free porcine DAO was tested in simulated intestinal fluid and showed a half-life of approximately 19 minutes. For the in vitro reduction of about 90% of histamine, a total of 50 nanokatal (nkat) free porcine DAO was required. This corresponded to the amount of enzyme isolated from about 100 g of pig kidney. The dietary supplement, containing a pig kidney extract did not show DAO activity. Instead, the histamine used in the experiment (0.75 mg) was apparently reduced by the adsorption to a capsule component by 18.9±2.3% within 5 h. Although the capsule preparation retained its overall structure and shape for at least 90 min in simulated gastric fluid, the apparent histamine reduction was significantly reduced to 12.1±2.3% (P≤0.05). Thus, an alternative to porcine DAO is urgently needed to provide adequate supplementation for histamine-intolerant individuals.

In this context, microbial DAOs can be easily produced in large quantities in suitable expression systems. For this purpose, some microbial DAOs have been studied and characterized in the art. Microbial DAOs exhibit varying properties resulting in certain advantages and disadvantages with regard to the degradation of biogenic amines from food. For example, using a histamine oxidase from Arthrobacter crystallopoietes KAIT-B-007, histamine can be degraded with a high kinetic efficiency. However, this enzyme, as well as, for example, an amine oxidase from Aspergillus niger, is not suitable to degrade the biogenic amines putrescine, cadaverine or spermidine.

Accordingly, the technical problem underlying the present invention is the provision of improved means for the degradation of a broad range of biogenic amines in vitro and in vivo.

The solution to the above technical problem is achieved by the embodiments characterized in the claims.

In particular, in one aspect, the present invention relates to an enzyme having diamine oxidase activity for use in medicine, wherein said enzyme comprises
(i) the amino acid sequence of SEQ ID NO: 1; or
(ii) an amino acid sequence having at least 70% sequence identity to SEQ ID NO: 1 and having diamine oxidase activity.

In preferred embodiments, the above enzyme consists of
(i) the amino acid sequence of SEQ ID NO: 1; or
(ii) the amino acid sequence having at least 70% sequence identity to SEQ ID NO: 1 and having diamine oxidase activity.

In a specific embodiment, the above enzyme consists of the amino acid sequence of SEQ ID NO: 1.

As used herein, the term “enzyme having diamine oxidase activity” relates to an enzyme that catalyzes the oxidation of biogenic amines according to the general reaction

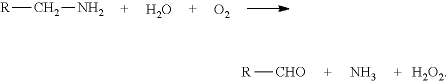

The enzyme having diamine oxidase activity used in the present invention is either (i) the Yarrowia lipolytica PO1f diamine oxidase-1 (DAO-1) having the amino acid sequence of SEQ ID NO: 1, the advantageous use of which in the degradation of biogenic amines has been discovered in the present invention, or (ii), in the case of an enzyme having at least 70% sequence identity to SEQ ID NO: 1 and having diamine oxidase activity, a respective DAO derived therefrom.

The term “enzyme comprising an amino acid sequence having at least 70% sequence identity to SEQ ID NO: 1 and having diamine oxidase activity” relates to polypeptides that can comprise any number of amino acid substitutions, additions, or deletions with respect to the amino acid sequence of SEQ ID NO: 1, e.g. 1, 2, 3, 4, 5, 6, 7, 8, 9, 10, or any number up to 200 (i.e., any integer n, wherein 1≤n≤200), amino acid substitutions, additions, or deletions with respect to the amino acid sequence of SEQ ID NO: 1, provided that the resulting polypeptides fulfil the requirement of having at least 70% sequence identity to SEQ ID NO: 1 and retain biological activity of a diamine oxidase. In this context, the term “retains the biological activity of a diamine oxidase” as used herein relates to polypeptides that have at least 5%, at least 10%, at least 15%, at least 20%, at least 25%, at least 30%, at least 35%, at least 40%, at least 45%, at least 50%, preferably at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 97.5%, at least 98%, at least 98.5%, at least 99%, at least 99.5%, 100%, or more than 100% of the activity of the Y. lipolytica PO1f DAO-1 having the amino acid sequence of SEQ ID NO: 1, as determined in a standard diamine oxidase activity assay known in the art.

While the number of amino acid substitutions, additions, or deletions is generally only limited by the above proviso concerning the sequence identity, biological activity of the resulting polypeptide, and absolute number of amino acid substitutions, additions or deletions, it is preferable that the resulting polypeptide has at least 72%, at least 74%, at least 76%, at least 78%, at least 80%, at least 82%, at least 84%, at least 86%, at least 88%, at least 90%, at least 91%, at least 92%, at least 93%, at least 94%, at least 95%, at least 95.5%, at least 96%, at least 96.5%, at least 97%, at least 97.5%, at least 98%, at least 98.2%, at least 98.4%, at least 98.6%, at least 98.8%, at least 99%, at least 99.1%, at least 99.2%, at least 99.3%, at least 99.4%, at least 99.5%, at least 99.55%, at least 99.6%, at least 99.65%, at least 99.7%, at least 99.75%, at least 99.8%, at least 99.85%, at least 99.9%, at least 99.91%, at least 99.92%, at least 99.93%, at least 99.94%, at least 99.95%, at least 99.96%, at least 99.97%, at least 99.98%, or at least 99.9% sequence identity to SEQ ID NO: 1.

Means for determining the sequence identity of an amino acid sequence to a reference sequence are known in the art.

In specific embodiments, the above enzyme DAO-1 can be purified from Y. lipolytica. In other specific embodiments, the above enzyme can be recombinantly produced in suitable host cells, e.g., in suitable microbial host cells as known in the art, or in suitable yeast host cells as known in the art. Suitable yeast host cells in this respect include Y. lipolytica and Komagataella phaffii, wherein K. phaffii is particularly preferred. Means for the recombinant expression of the above enzyme in suitable host cells are not particularly limited and are known in the art.

In specific embodiments, the above enzyme is for use in a method of preventing or treating a condition or disease that is associated with increased levels of biogenic amines in a subject, preferably a mammalian subject, more preferably a human subject.

In this context, the term “increased levels of biogenic amines” as used herein refers to increased levels of biogenic amines in vivo, e.g., in the small intestine, where biogenic amines first accumulate before passing into the bloodstream, or in the blood of the subject. Means for determining the level of biogenic amines in a subject are not particularly limited and are known in the art.

Increased in vivo levels of biogenic amines can result from pathological conditions in the body, e.g., dysregulated immune functions as in allergic reactions, or can result from the ingestion of biogenic amines by the subject, e.g., by consuming foodstuffs containing high levels of biogenic amines.

In specific embodiments, the condition or disease that is associated with increased levels of biogenic amines is selected from the group consisting of allergies, acute and chronic allergic diseases, allergic reactions, allergy-like reactions, itching (pruritus), diarrhea, redness (erubescence), vomiting (emesis), acute and chronic biogenic amine poisoning, hypotonia, difficulty of breathing, biogenic amine intolerance, anaphylaxis, anaphylactic shock, acute and chronic urticaria, asthma, hay fever, allergic rhinitis, allergic conjunctivitis, headache, migraine, atopic dermatitis, mastocytosis, mast cell activation syndrome (MCAS), pre-eclampsia, hyperemesis gravidarum, pre-term labor, peptic ulcers, acid reflux, sepsis, fibromyalgia, chronic fatigue syndrome, and spondylitis.

The term “biogenic amines” as used herein relates to any amine compound that is a constituent of, secreted by or a metabolite of a plant, animal, fungus, or microorganism, provided that said amine can have an adverse effect on a subject. However, in preferred embodiments, the biogenic amine is selected from the group consisting of histamine, tyramine, putrescine, cadaverine, agmatine, spermidine, and tryptamine, preferably from the group consisting of histamine, tyramine, putrescine, and cadaverine. In specific embodiments, the biogenic amine is histamine.

In a related aspect, the present invention relates to a method of preventing or treating a condition or disease that is associated with increased levels of biogenic amines in a subject, comprising the step of administering an enzyme having diamine oxidase activity to the subject, wherein said enzyme comprises
(i) the amino acid sequence of SEQ ID NO: 1; or
(ii) an amino acid sequence having at least 70% sequence identity to SEQ ID NO: 1 and having diamine oxidase activity.

In this aspect, all relevant definitions and limitations indicated above for the enzyme for use of the present invention apply in an analogous manner. In particular, the enzyme as such, the condition or disease that is associated with increased levels of biogenic amines in a subject, the subject, and the biogenic amines are as defined above.

In this context, dosages, dosage regimens, administration modes and suitable formulations for the enzymes used in the present invention are not particularly limited and are known to the person skilled in the art and/or can be easily determined by the person skilled in the art.

In a further aspect, the present invention relates to the use of an enzyme having diamine oxidase activity in the production of a biogenic amine-depleted product, wherein said enzyme comprises
(i) the amino acid sequence of SEQ ID NO: 1; or
(ii) an amino acid sequence having at least 70% sequence identity to SEQ ID NO: 1 and having diamine oxidase activity.

In this aspect, all relevant definitions and limitations indicated above for the enzyme for use of the present invention apply in an analogous manner. In particular, the enzyme as such and the biogenic amines are as defined above.

Means of using the enzyme used in the present invention in the production of a biogenic amine-depleted product are not particularly limited and are known in the art. Respective means include for example the step of contacting (i) a biogenic amine-containing product, and/or (ii) a biogenic amine-containing intermediate product of said product, with said enzyme under conditions and for a duration of time suitable to degrade a biogenic amine present in said product and/or in said intermediate product of said product. Respective conditions and or durations of time are not particularly limited and are known to the person skilled in the art and/or can be easily determined by the person skilled in the art. These include for example the incubation of the product or intermediate product thereof with the enzyme in an amount providing an enzyme activity of 0.1 nkat/mL or 0.1 nkat/mg of the product or intermediate product thereof within at most 5 h at a temperature of 37±1° C. and in a pH range of pH 6 to 8.

The term “biogenic amine-depleted product” as used herein refers to a product whose biogenic amine content has been reduced with respect to its original biogenic amine content. In specific embodiments, the biogenic amine content has been reduced by at least 50%, at least 55%, at least 60%, at least 65%, at least 70%, at least 75%, at least 80%, at least 85%, at least 90%, at least 95%, at least 95.5%, at least 96%, at least 96.5%, at least 97%, at least 97.2%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9%.

Products amenable to the above use according to the present invention are not particularly limited and include any products containing biogenic amines in which the reduction of biogenic amine content might be of interest. Preferably, the product is selected from the group consisting of foodstuffs and feedstuffs. In this context, the term “foodstuffs” as used herein refers to any solid or liquid comestibles, e.g., foods and drinks, that can be ingested by a human for nutritional and/or recreational purposes. Likewise, the term “feedstuffs” as used herein refers to any solid or liquid comestibles, e.g., feeds, that can be ingested by an animal for nutritional purposes. In specific embodiments, the above product is a foodstuff, selected from the group consisting of fermented foodstuffs, cheeses, sauerkraut, sausages, wine, chocolate, yeast extracts, fish and fish products, raw meats, vegetables, dairy products and fresh milk.

In a related further aspect, the present invention relates to a method for the production of a biogenic amine-depleted product, comprising the step of contacting (i) a biogenic amine-containing product, and/or (ii) a biogenic amine-containing intermediate product of said product, with an enzyme having diamine oxidase activity under conditions and for a duration of time suitable to degrade a biogenic amine present in said product and/or in said intermediate product of said product, wherein said enzyme comprises
(i) the amino acid sequence of SEQ ID NO: 1; or
(ii) an amino acid sequence having at least 70% sequence identity to SEQ ID NO: 1 and having diamine oxidase activity.

In this aspect, all relevant definitions and limitations indicated above for the enzyme for use of the present invention and/or the use of the present invention apply in an analogous manner. In particular, the enzyme as such, the product, the term “biogenic amine-depleted product”, and the biogenic amines are as defined above.

Respective conditions and or durations of time applicable in the methods of the present invention are not particularly limited and are known to the person skilled in the art and/or can be easily determined by the person skilled in the art. These include for example the incubation of the product or intermediate product thereof with the enzyme as defined above for the uses of the present invention.

In a further aspect, the present invention relates to a product obtained by the above method.

In this aspect, all relevant definitions and limitations indicated above for the enzyme for use of the present invention, the use of the present invention, and/or the method of the present invention apply in an analogous manner. In particular, the enzyme as such, the product, the term “biogenic amine-depleted product”, and the biogenic amines are as defined above.

In a further aspect, the present invention relates to a functional food, dietary supplement, or pharmaceutical composition, comprising an enzyme having diamine oxidase activity, wherein said enzyme comprises
(i) the amino acid sequence of SEQ ID NO: 1; or
(ii) an amino acid sequence having at least 70% sequence identity to SEQ ID NO: 1 and having diamine oxidase activity.

In this aspect, all relevant definitions and limitations indicated above for the enzyme for use of the present invention apply in an analogous manner. In particular, the enzyme as such is as defined above.

In this context, the term “functional food” as used herein refers to any foodstuffs, e.g., any solid or liquid comestibles, e.g., foods and drinks, that are “functionalized” by the addition of the enzyme used in the present invention, i.e., that are modified to exhibit the additional effect of being able to reduce the levels of biogenic amines in a subject, preferably a human subject, upon ingestion of the functional food.

Further, the term “dietary supplement” as used herein refers to any manufactured product intended to supplement a subject's diet, preferably a human subject's diet, in the form of a pill, capsule, tablet, powder or liquid, wherein the form of a tablet, e.g., a sucrose-based tablet is particularly preferred. Respective dietary supplements can be formulated together with an additional agent, e.g., a catalase.

Particular foodstuffs, forms of dietary supplements, forms of pharmaceutical compositions, dosages, dosage regimens, and suitable formulations for the enzymes used in the present invention are not particularly limited and are known to the person skilled in the art and/or can be easily determined by the person skilled in the art.

Of note, the biogenic amine-depleted products, functional foods, dietary supplements, and pharmaceutical compositions of the present invention can be produced without the use of any animal-derived products, so that the same can fulfill vegetarian and vegan standards.

In a final aspect, the present invention relates to the use of an enzyme having diamine oxidase activity in a functional food or in a dietary supplement, wherein said enzyme comprises
(i) the amino acid sequence of SEQ ID NO: 1; or
(ii) an amino acid sequence having at least 70% sequence identity to SEQ ID NO: 1 and having diamine oxidase activity.

In this aspect, all relevant definitions and limitations indicated above for the enzyme for use of the present invention. as well as for the functional food or dietary supplement of the present invention, apply in an analogous manner. In particular, the enzyme as such, the functional food, and the dietary supplement are as defined above.

As used herein, the term “comprising” “comprises” expressly includes the terms “consisting essentially of”/“consists essentially of” and “consisting of”/“consists of”, i.e., all of said terms are interchangeable with each other herein.

Further, as used herein, the term “about” represents a modifier of ±10% of the specified value, preferably ±7.5%, ±5%, ±3%, ±2%, or ±1% of the specified value. Thus, by way of example, the term “about 10” includes the ranges 9 to 11, 9.25 to 10.75, 9.5 to 10.5, 9.7 to 10.3, 9.8 to 10.2, and 9.9 to 10.1.

The diversity of enzymes found in microorganisms could have the potential of providing a competitive DAO with similar or even better attributes compared to e.g., porcine DOA as known in the art. If administered in a dietary supplement or generally used in the food industry, an advantage of microbial DAOs is the cost-effective and convenient enzyme production. Accordingly, the present invention identified Yarrowia lipolytica (Y. lipolytica) PO1f as a promising producer of an alternative microbial DAO. This yeast represents one of the yeast species most frequently found in raw milk and is also found in various types of cheese. It contributes to the development of flavor and aroma during the ripening of the cheese due to its proteolytic and lipolytic activity and is, therefore, a desirable and important constituent of the product. The yeast occurs naturally in cheese since contamination can happen at different production stages (raw milk, air, brine, contact with surfaces) and it grows well under the common environmental conditions in foods. However, Y. lipolytica was classified as an opportunistic pathogen in 2018 by the European Food Safety Authority and was given the qualified presumption of safety status only for production purposes. This means that the yeast should not be present in the final food as viable cells but is allowed to be used as an inactivated biomass as a novel food. Y. lipolytica was identified as a biogenic amine producer but was also suspected of being capable of degrading biogenic amines during the ripening of the food. However, the enzymes responsible for this degradation of biogenic amines had not been identified or described prior to the present invention.

According to the present invention, the bioreactor cultivation of the genetically modified Yarrowia lipolytica PO1f achieved a specific DAO activity of 1301±54.2 nkat/g_{Protein}, which corresponded to a 93-fold increase in specific DAO activity compared with native DAO-1 production in Y. lipolytica PO1f and a 145-fold increase in specific DAO activity compared with DAO extraction from porcine kidney. Subsequent workup including chromatographic purification yielded a specific DAO activity of 4738±31 nkat/g_{Protein}, which corresponded to a nearly 60-fold increase in specific DAO activity compared with extracted and purified DAO from porcine kidney. One liter of bioreactor cultivation generated approximately the same DAO activity obtained after extraction and partial purification from 5 kg of pig kidney. Moreover, the microbial production of DAO-1 requires much less work than extraction from pig kidney.

In this context, porcine DAO preparations as known in the art, especially in food supplements, were proven to be inactive DAO preparations. Furthermore, the extraction and partial purification of porcine DAO in the art yielded a total activity of 50 nkat from 100 g pig kidney with a low specific DAO activity of 81 nkat/g_{Protein}. In contrast, the production of DAO-1 in Komagataella phaffii described herein yielded 50.000 nkat/L_{Medium} with a specific DAO-1 activity of 2500 nkat/g_{Protein}. As demonstrated, the DAO-1 can be further purified 7.2-fold, which would yield a specific DAO-1 activity of the DAO-1 produced in Komagataella phaffii of 18.000 nkat/g_{Protein}. This is more than 200-fold higher than the specific DAO activity obtained from porcine kidney. As also described herein, the DAO-1 was evaluated for its histamine degradation capability under simulated intestinal conditions, whereby 690 nkat DAO-1 was formulated in a tablet and degraded a total of 22 mg histamine. Thereby, it can be concluded that DAO-1 according to the present invention is capable of degrading relevant amounts of histamine in vivo. This total activity (690 nkat) was obtained in the bioreactor cultivation of Komagataella phaffii in a 14 mL cell suspension. For the DAO-1 tablet, 44 mg of protein were formulated to attain the desired DAO-1 activity.

For the preparation of an equivalent tablet using the partially purified porcine DAO extract, around 1.4 kg of porcine kidney would be required to apply the same total activity (690 nkat). With an average weight of 150 g per kidney (for pigs of 30 kg), almost 10 kidneys and therefore 5 pigs would be required to prepare one equivalent tablet. With a specific DAO activity of 81 nkat/g_{Protein}, more than 8 g of protein have to be formulated in the tablet.

In conclusion, the microbial production of DAO is inevitable for the preparation of a satisfactory DAO-containing tablet. The extraction of DAO from pig kidney for the preparation of DAO tablets is from the technical, economical, and ecological viewpoint not reasonable.

Regarding the substrate selectivity, DAO-1 according to the present invention and porcine DAO both catalyze the oxidative deamination of histamine, putrescine, cadaverine and spermidine. However, the substrates tryptamine and tyramine seem to be poor substrates for porcine DAO. In contrast, DAO-1 efficiently deaminates both substrates, which is an advantage of the DAO-1 according to the present invention over porcine DAO regarding its substrate selectivity. In this context, tyramine and tryptamine are considered being one of the most commonly found biogenic amines in foods.

DAO-1 was investigated herein in vitro studies with regard to the efficiency of histamine degradation. The initial 150 mg/L (100%) of histamine used was degraded within 5 hours (37° C.) to 37.4±+0.65 mg/L (25±1.7%). Although compared to porcine DAO, DAO-1 according to the present invention has a lower affinity for the substrate histamine, DAO-1 showed similar satisfactory histamine degradation in vitro.

DAO-1 exhibits the peculiarity among microbial DAOs that, in addition to histamine, it also degrades other relevant biogenic amines such as tyramine, putrescine, cadaverine, agmatine, spermidine, and tryptamine. Since these biogenic amines are also present in fermented foods and, like histamine, have toxicological properties for humans, it is advantageous if they can be degraded simultaneously by an enzyme. DAO from the pig kidney is also able to degrade histamine, putrescine and cadaverine. However, the biogenic amine tyramine is not metabolized by this enzyme. Thus, the somewhat more efficient histamine degradation by DAO from the porcine kidney is outweighed by the advantages of the more efficient and simpler production of DAO-1 and the broad substrate spectrum.

In summary, the present invention provides uses of a Yarrowia lipolytica PO1f-derived diamine oxidase (DAO-1) that is advantageously and unexpectedly characterized by (i) superior specific activity, (ii) good in vitro degradation of biogenic amines such as histamine, and (iii) a broad substrate selectivity. Further, the DAO-1 enzyme used in the present invention can advantageously be produced in a much simpler, faster, more efficient, and economic manner. Furthermore, the use of a yeast-derived DAO as opposed to e.g., animal-derived DAO poses less problems with respect to ethical aspects, as well as user acceptance and compliance.

The figures show:

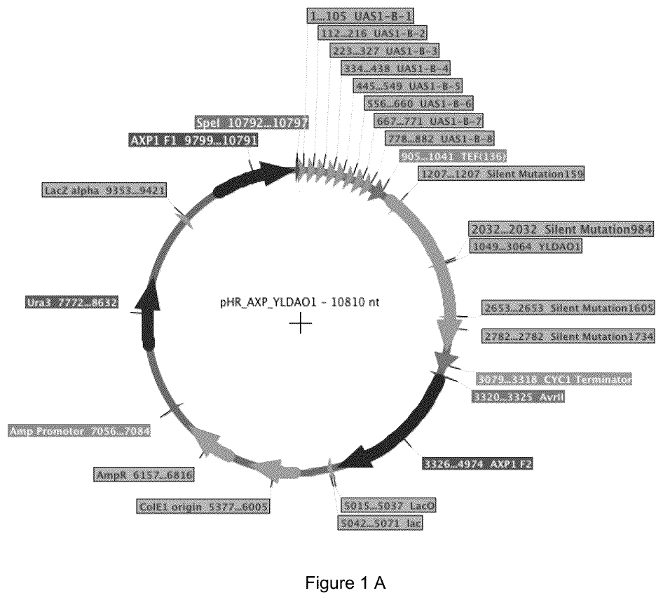

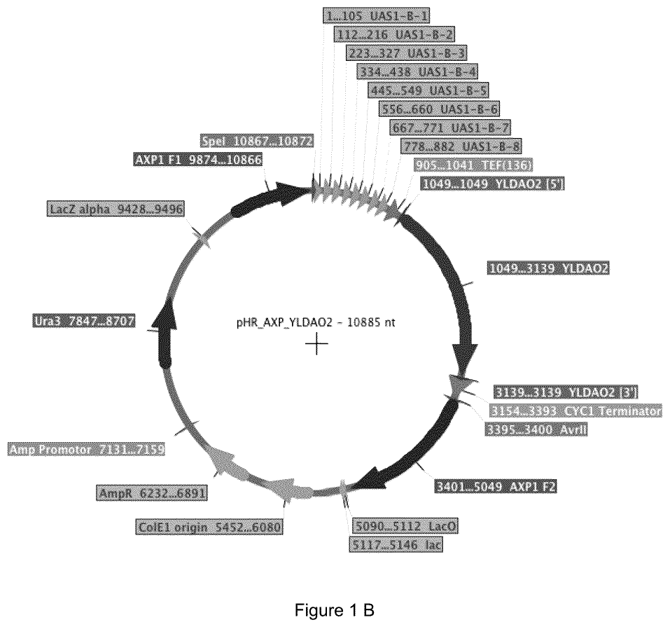

FIG. 1:
(A) pHR_axp_dao-1 plasmid for the integration of dao-1 into the genome (axp locus) of Y. lipolytica PO1f by homologous recombination. (B) pHR_axp_dao-2 plasmid for the integration of dao-2 into the genome (axp locus) of Y. lipolytica PO1f by homologous recombination.

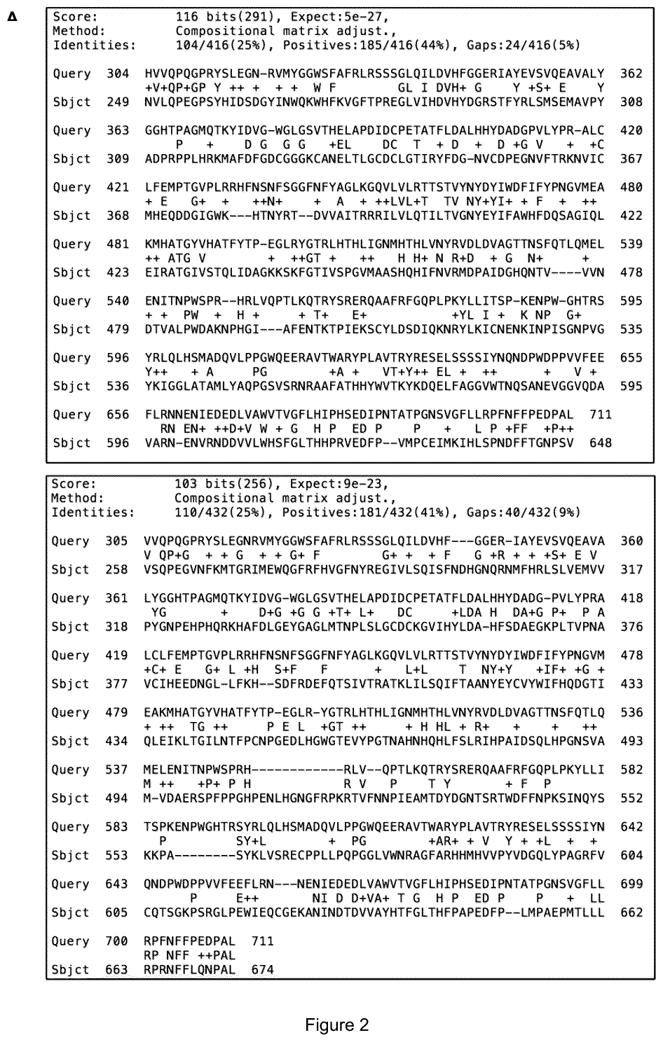

FIG. 2:
BLASTP amino acid sequence alignment of porcine DAO with the two putative DAOs DAO-1 (A; Uniprot: Q6CGT2) and DAO-2 (B; Uniprot: A0A371BXN9) from Y. lipolytica PO1f.

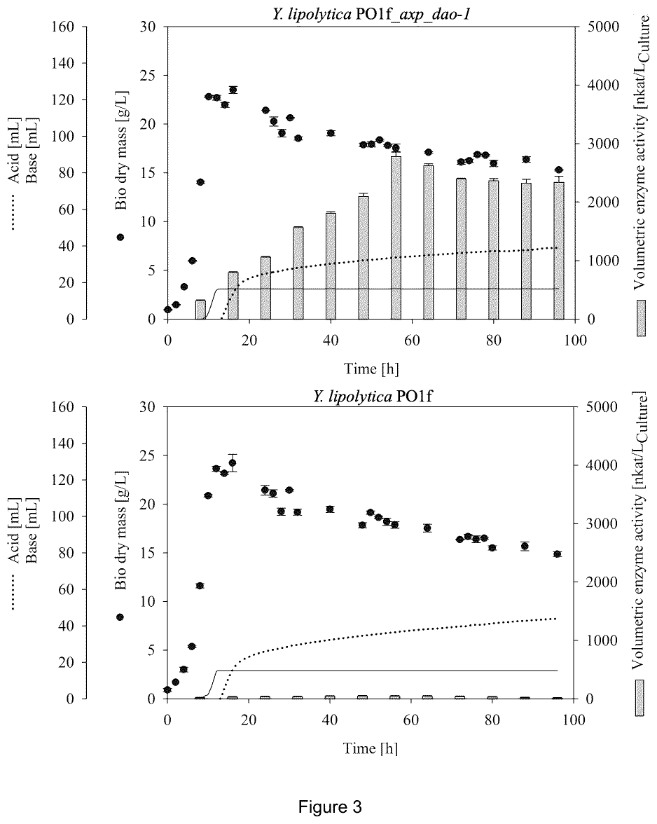

FIG. 3:
Bioreactor cultivation of Y. lipolytica PO1f_axp_dao-1 and the reference strain Y. lipolytica PO1f in YPD_{Ura} medium. Working volume: 0.8 L, 28° C., pH 6.5.

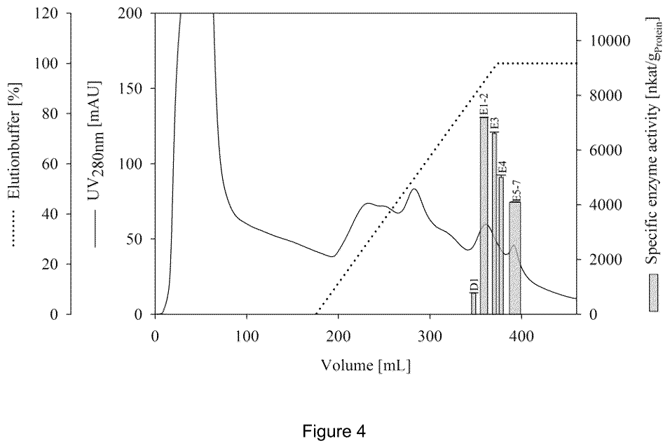

FIG. 4:
Hydrophobic interaction chromatography purification of DAO-1. Column material: Toyopearl phenyl 650M (CV=22 mL).

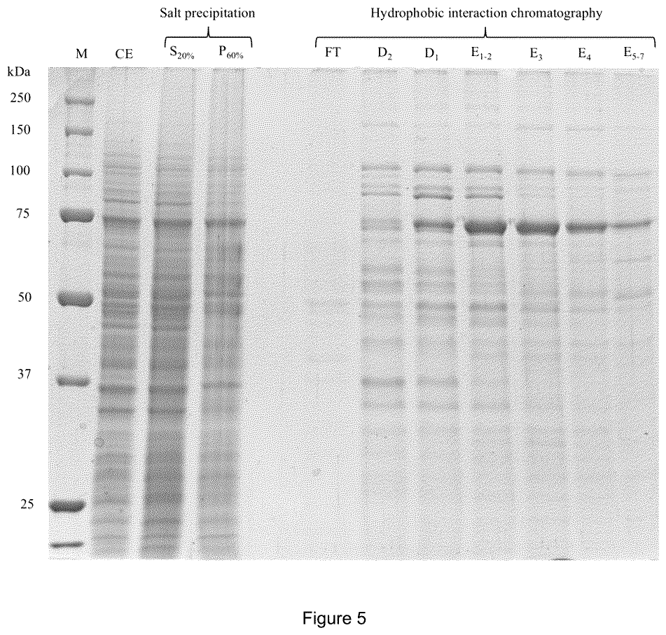

FIG. 5:
SDS-PAGE analysis of the DAO-1 purification by salt precipitation and hydrophobic interaction chromatography. Precision Plus Protein™ unstained protein standard 10-250 kDa (M); Crude extract after disruption (CE); Supernatant after 20% (w/v) salt precipitation (S20%); Suspended pellet after 60% (w/v) salt precipitation (P60%), HIC flow through (FT); HIC elution fractions (D2-E7).

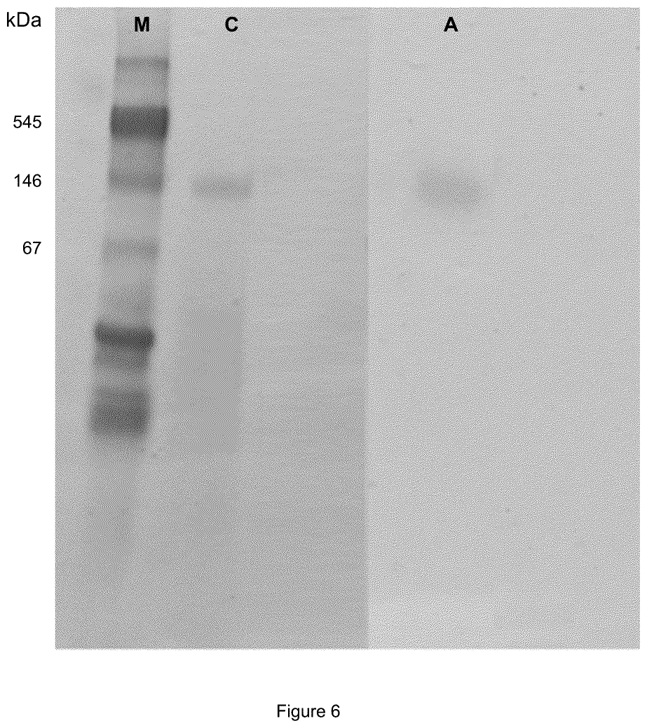

FIG. 6:
Native-PAGE analysis of the purified DAO-1. Marker (M): Serva native marker (21-720 kDa); 5 μg_{Protein} per lane; stained with Coomassie Brilliant Blue G-250 (C) and active (A) using histamine as substrate.

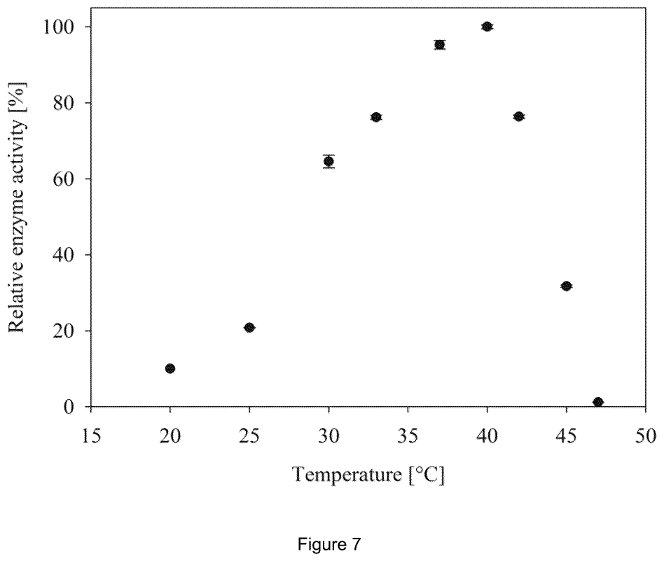

FIG. 7:
Influence of temperature on the maximal activity of DAO-1. DAO-1 activity measured with the DA-67 assay in PIPES buffer (25 mM; pH 7.2). Maximal DAO-1 activity (100%)=1.88±0.01 nkat/mL.

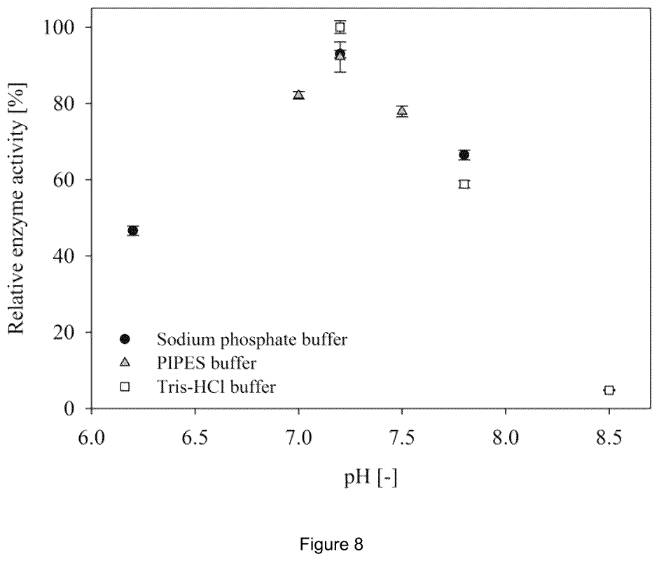

FIG. 8:
Influence of the pH-value and buffer on the maximal activity of DAO-1. DAO-1 activity measured with the DA-67 assay at 37° C. Maximal DAO-1 activity (100%)=1.84±0.03 nkat/mL.

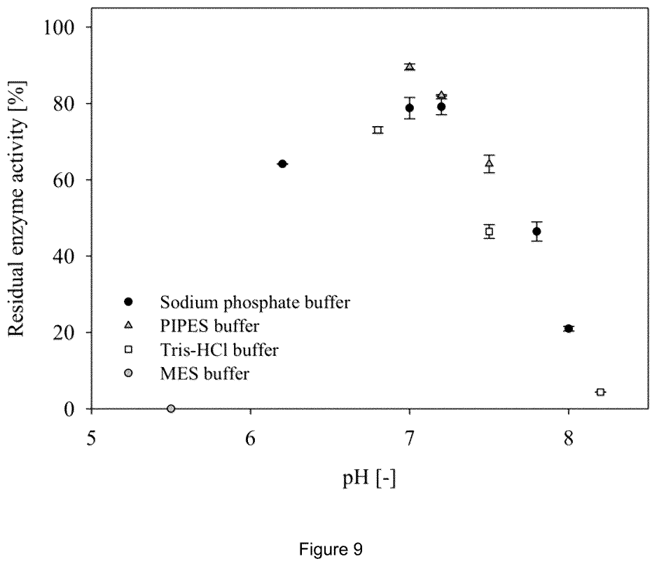

FIG. 9:
Residual activity of DAO-1 depending on buffer system (each 20 mM) and pH-value after 5 h at 37° C. DAO-1 activity measured with the DA-67 assay at 37° C. under standard assay conditions. Residual activities of each data point are compared to the initially applied DAO-1 activity and given in percent (100%=0.156±0.01 nkat/mL).

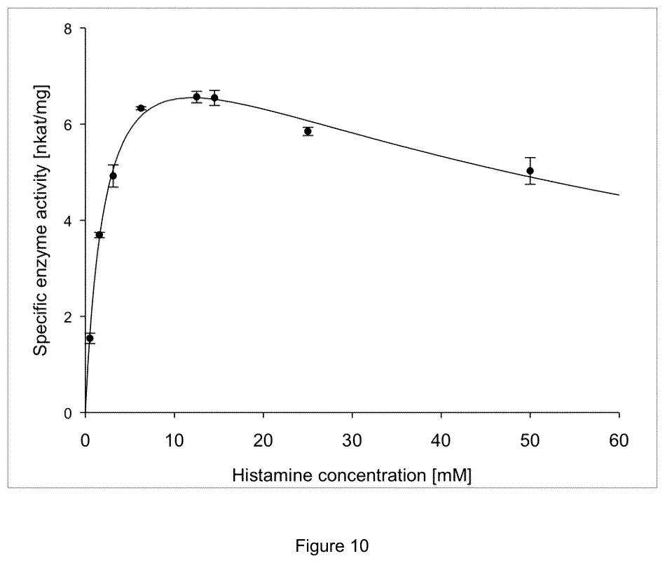

FIG. 10:
Michaelis-Menten kinetics of DAO-1. DAO-1 activity determined with the DA-67 assay in PIPES buffer (25 mM; pH 7.2) at 37° C.

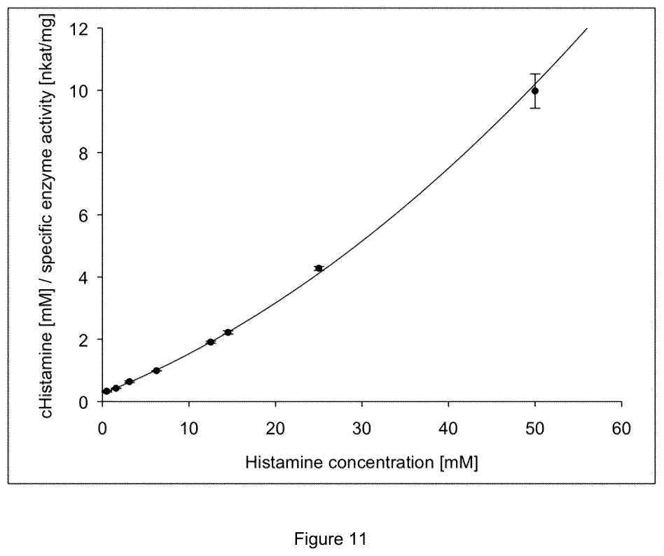

FIG. 11:
Michaelis-Menten kinetics of DAO-1 linearized according to Hanes-Woolf; enzyme activity determined with the DA-67 assay with histamine (0.5 mM-50 mM) as substrate at 37° C. in PIPES buffer (25 mM; pH 7.2).

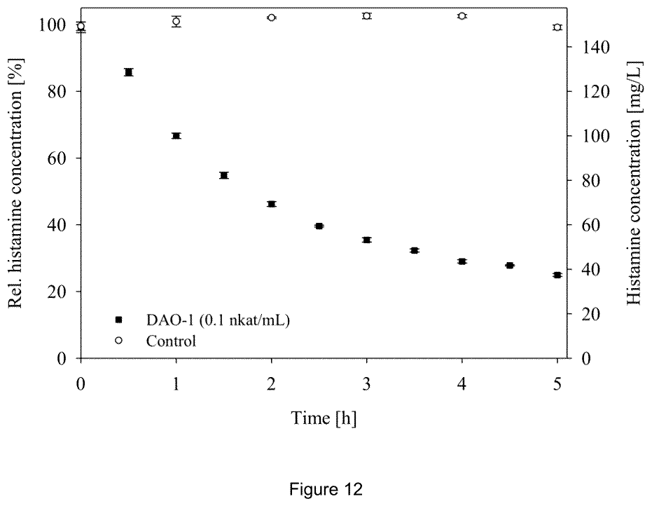

FIG. 12:
In vitro reduction of histamine in a food-relevant concentration (150 mg/L; 1.35 mM) using 0.1 nkat/mL of DAO-1 at 37° C.

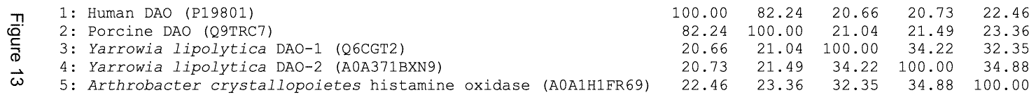

FIG. 13:
Percent identity matrix of different amine oxidases (created by Clustal2.1).

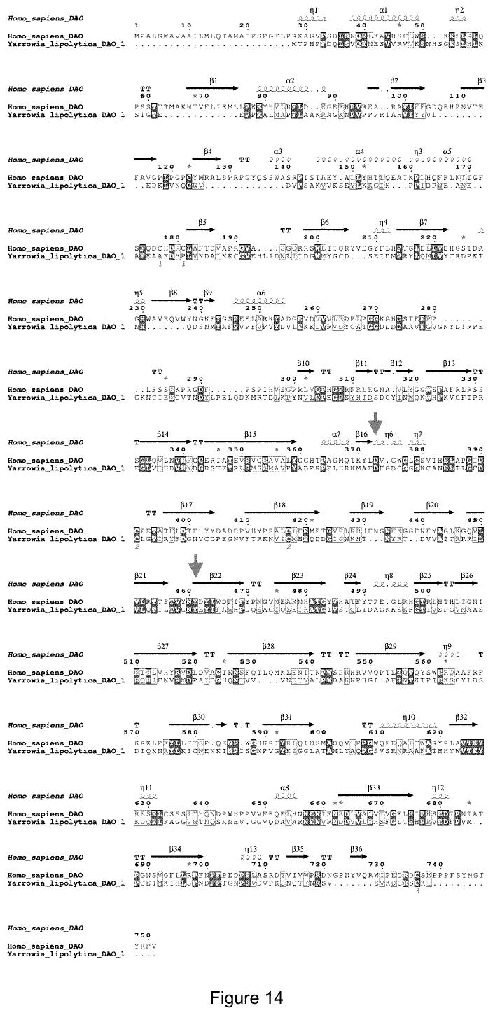

FIG. 14:
Alignment of the amino acid sequences of human DAO and the Y. lipolytica PO1f DAO-1. Blue arrows indicate the active sites (Positions 373 and 461) of human DAO. The alignment was done using ESPript (https://espript.ibcp.fr).

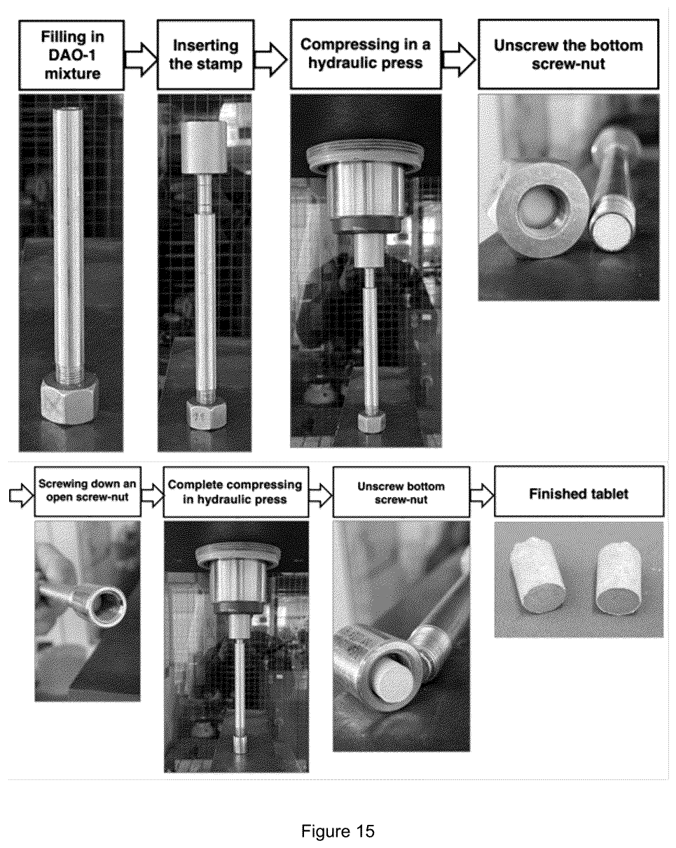

FIG. 15:
Preparation of DAO-1 tablets using a self-built tablet press.

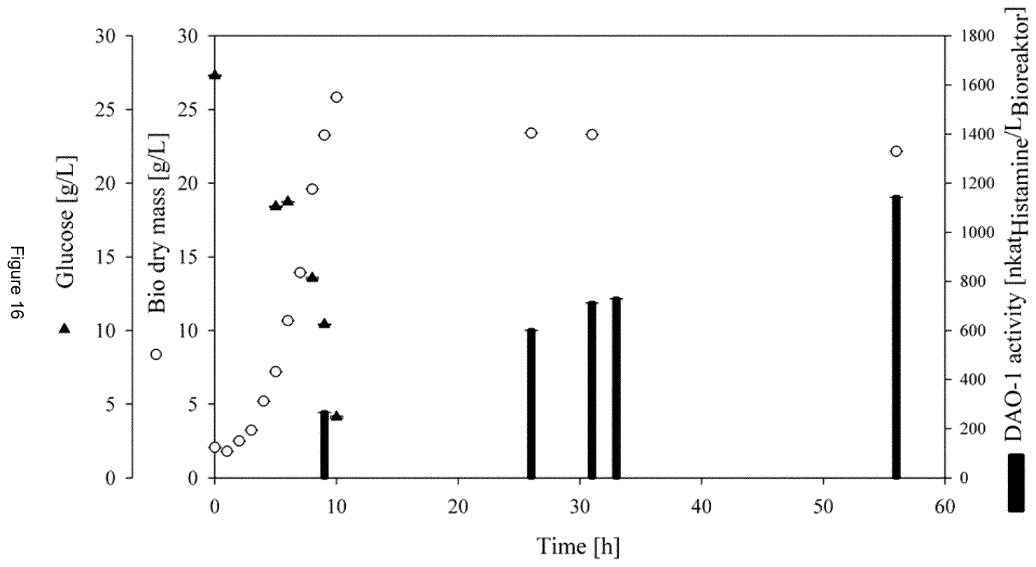

FIG. 16:
Bioreactor cultivation of Yarrowia lipolytica_axp_dao-1 in YPD_{Ura} media; working volume 5 L at 28° C. (pH 6.5). Y. lipolytica cells were disrupted for DAO-1 activity determinations using the TissueLyser II (Qiagen, Düsseldorf, Germany) with glass beads (0.75 mm in diameter). DAO-1 activity was determined under standard assay conditions using the DA-67 assay.

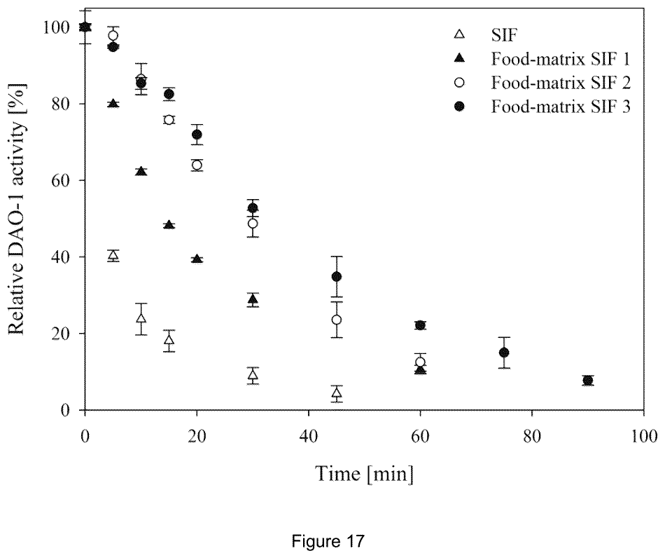

FIG. 17:
Stability of DAO-1 in pure SIF and supplemented with different food matrices. Food-matrix SIF 1 contains 50 g/L BSA and 25 g/L sucrose. Food-matrix SIF 2 contains BSA and WPI each at 25 g/L and 50 g/L sucrose. Food-matrix SIF 3 contains BSA, WPI, sodium-caseinate each at 16.67 g/L and 50 g/L sucrose. DAO-1 activity determined with the DA-67 assay under standard assay conditions (100% DAO-1 activity=1.25±0.15 nkat_{Histamine}/mL).

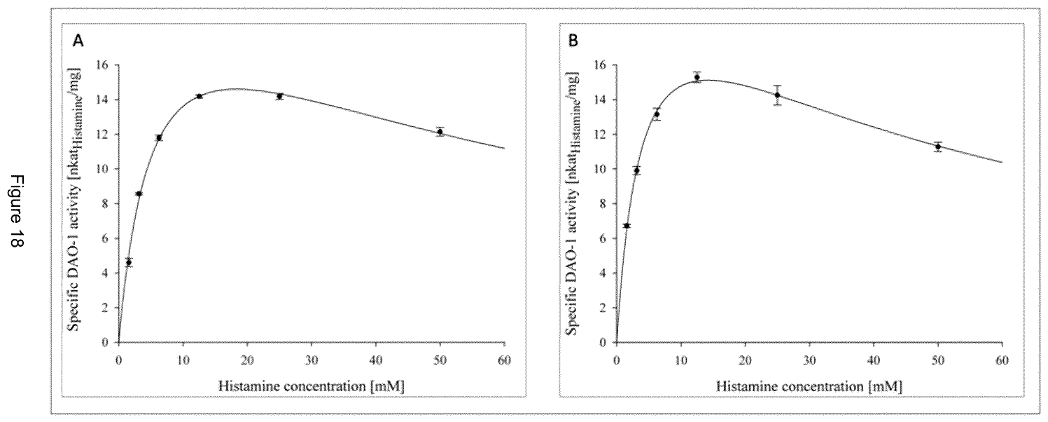

FIG. 18:
Michaelis-Menten kinetics of DAO-1 in unhydrolyzed (A) and hydrolyzed (B) food-matrix SIF 3. Histamine concentrations ranging from 1.56 to 50 mM. DAO-1 activity was determined with the DA-67 assay.

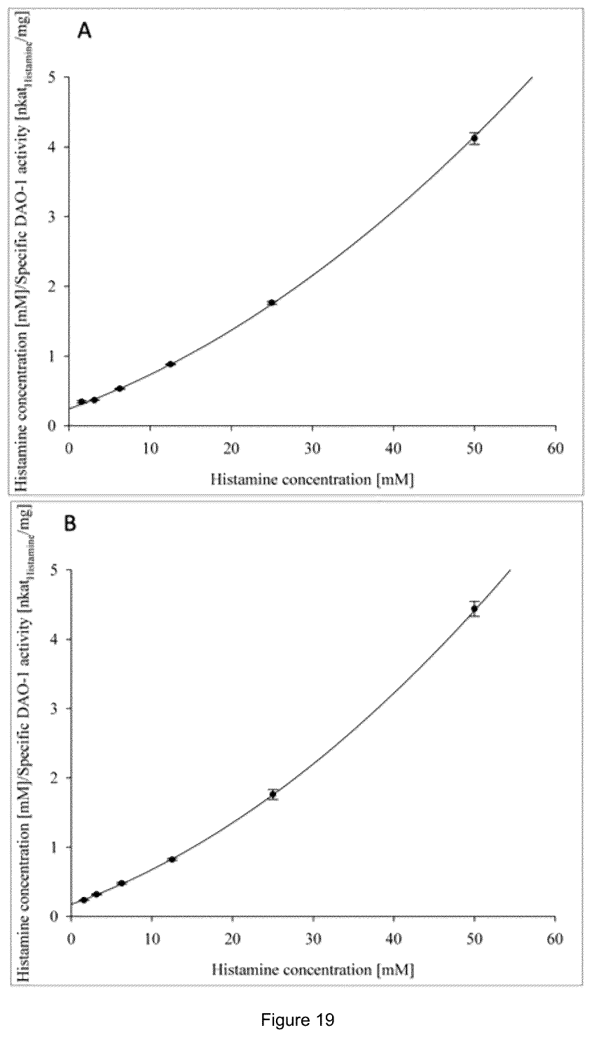

FIG. 19:
Michaelis-Menten kinetics of DAO-1 in a food-matrix simulated intestinal fluid under unhydrolyzed (A) and hydrolyzed (B) conditions at 37° C. Linearization as known in the art. Histamine concentration ranging from 1.56 to 50 mM.

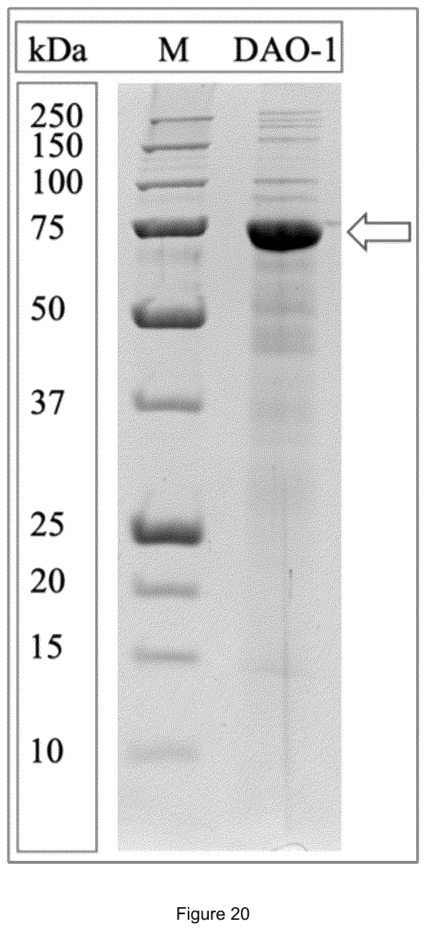

FIG. 20:
SDS-PAGE analysis of the DAO-1 preparation used for the preparation of DAO-1 tablets. Precision Plus Protein™ unstained protein standard 10-250 kDa (M). Arrow indicates the DAO-1 protein band.

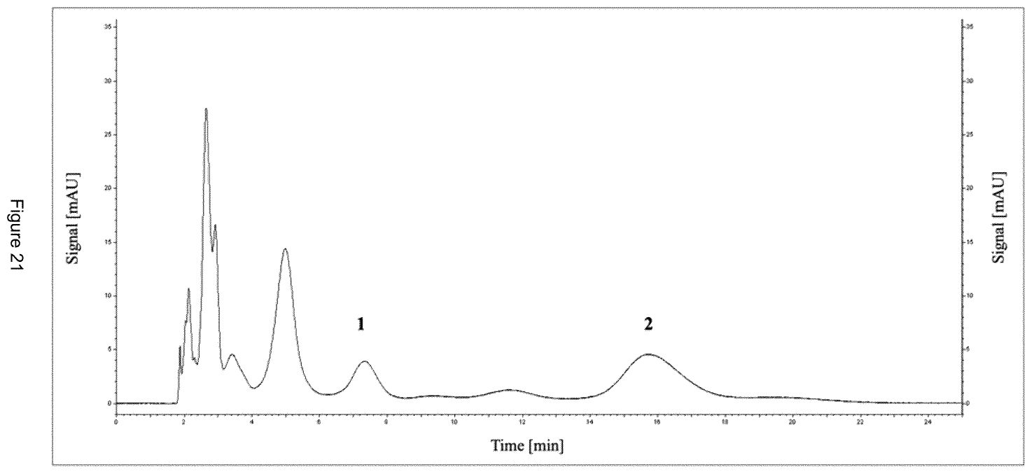

FIG. 21:
Example chromatogram (RP-HPLC) for the analysis of histamine (1) and thiamine (2) in the food-matrix SIF bioconversion sample (90 min) on the AquaC18 column (150×4.6 mm; 200 Å) from Phenomenex on the Platinblue UHPLC system from Knauer (Berlin, Germany). Detection at 210 nm.

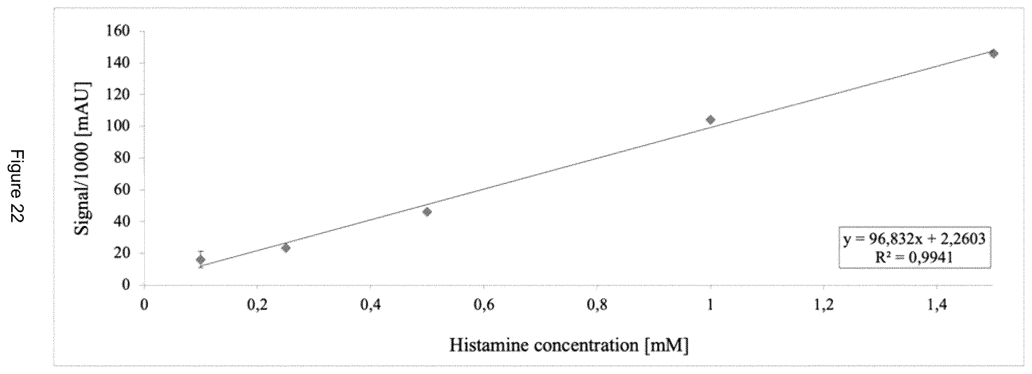

FIG. 22:
Exemplary histamine calibration for the quantification of histamine in the food-matrix SIF bioconversion samples (90 min) on the AquaC18 column (150×4.6 mm; 200 Å) from Phenomenex (Platinblue UHPLC system from Knauer (Berlin, Germany)). Detection at 210 nm.

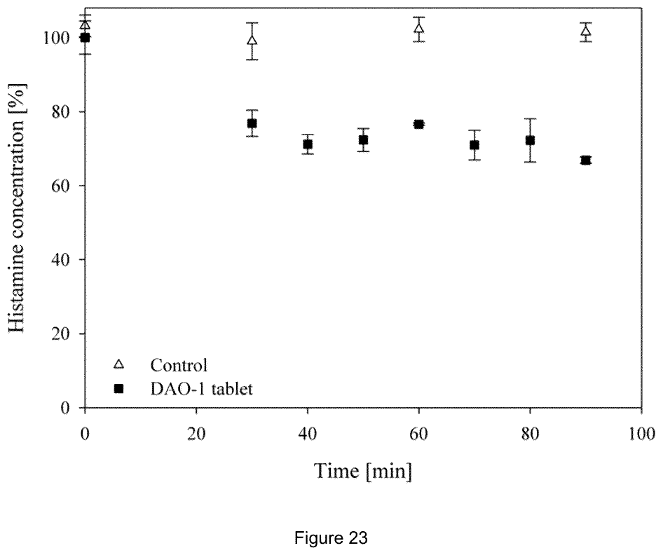

FIG. 23:
Bioconversion of 75 mg histamine in food-matrix SIF 3 by one DAO-1 tablet (690 nkat DAO-1). For the control no DAO-1 tablet was applied. Histamine concentrations were determined by RP-HPLC.

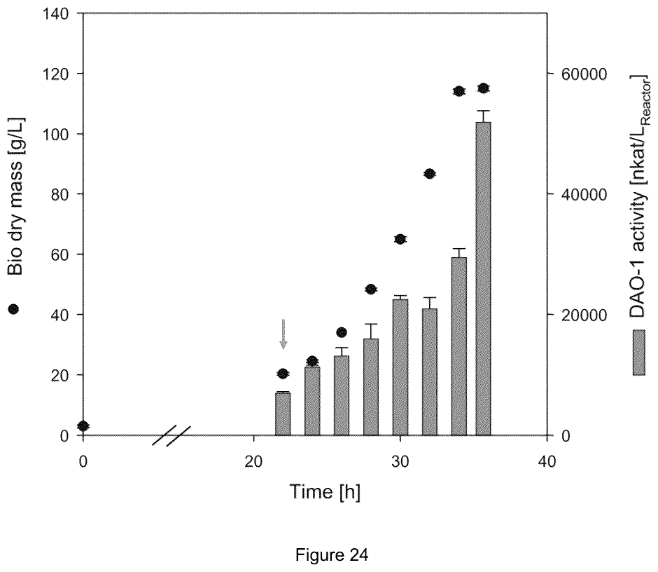

FIG. 24:
Cultivation process of the fed-batch bioreactor cultivation of Komagataella phaffii for the production of DAO-1. Arrow=start of the exponential glucose feed.

The present invention will be further illustrated by the following examples without being limited thereto.

### EXAMPLES

### Material and Methods

### Materials and Reagents

Disodium hydrogen phosphate (Na₂HPO₄), ammonium sulfate ((NH₄)₂SO₄), 1,4-piperazinediethanesulfonic acid (PIPES), tris-hydrogen chloride (—HCl), histamine dihydrochloride, sodium chloride (NaCl), sodium hydroxide (NaOH), 2-propanol, D(+)-sucrose and hydrogen peroxide 30% were purchased from Carl Roth GmbH (Karlsruhe, Germany). Sodium dihydrogen phosphate, sodium diethyldithiocarbamate, ortho-phosphoric acid (H₃PO₄) and thiamine chloride dihydrochloride were purchased from Merck (Darmstadt, Germany). Bovine serum albumin (BSA; modified Cohn Fraction V, pH 5.2) and Serva native marker (21-720 kDa) were purchased from Serva electrophoresis GmbH (Heidelberg, Germany). Bacto™ peptone, BD™ Difco™ Yeast nitrogen base and T4 DNA ligase were purchased from Thermo Fisher Scientific (Waltham, USA). Yeast Synthetic Drop-out Medium Supplements (without uracil, leucine, and tryptophan), Antifoam 204, phenylmethylsulfonylfluoride (PMSF), histamine (analytically pure), cadaverine, putrescine dihydrochloride, spermidine trihydrochloride, tryptamine, tyramine hydrochloride and catalase (from Micrococcus lysodeikticus; 111700 U/mL) were purchased from Sigma-Aldrich (Merck) (St. Louis, USA). Agmatine dihydrochloride was purchased from Synthonix Inc. (Wake Forest, USA). Uracil was purchased from Alfa Aesar (Haverhill, USA). (10-(carboxymethyl-aminocarbonyl)-3,7-bis(dimethylamino) phenothiazine sodium salt (DA-67) was purchased from Fujifilm Wako Chemicals U.S.A. Corp (Richmond, USA). Horseradish peroxidase (Grade I) was purchased from AppliChem GmbH (Darmstadt, Germany). Q5® High-Fidelity DNA polymerase and the restriction enzymes BssHII and NheI were purchased from New England Biolabs GmbH (Ipswich, USA). Precision Plus Protein™ unstained protein standard 10-250 kDa was purchased from Bio-Rad laboratories GmbH (Feldkirchen, Germany). The resin material Toyopearl Phenyl-650M was purchased from Tosoh Bioscience Inc. (San Francisco, USA).

### Strains and Media

The genes for the putative DAOs and plasmid propagation were cloned in Escherichia coli XL-1, grown in lysogeny broth media with 100 μg/mL ampicillin.

Yarrowia lipolytica PO1f was obtained from the International Centre for Microbial Resources and cultivated in yeast extract peptone dextrose (YPD) media (10 g/L yeast extract, 20 g/L Bacto™ peptone, 20 g/L glucose). Screening for positive transformants of the homologous recombinant integration of dao-1 and dao-2 in Y. lipolytica PO1f was done on selective agar which contained 6.7 g/L yeast nitrogen base (BD™ Difco™), 0.9 g/L CSM (Yeast Synthetic Drop-out Medium Supplements without uracil, leucine, and tryptophan), 20 g/L glucose and 15 g/L agar.

Cloning of the Dao Genes into the Vector for Homologous Recombination (pHR)

Genomic DNA of Y. lipolytica PO1f was isolated by mechanical cell disruption using glass beads (0.5-0.7 mm in diameter) and subsequent DNA extraction in ROTI®Phenol/Chloroform/Isoamyl as known in the art. Y. lipolytica PO1f was grown in 5 mL YPD in test tubes for 20 h at 30° C., stirred at 180 rpm and was completely harvested for DNA isolation.

The genes coding for the two putative DAOs in Y. lipolytica PO1f (SEQ ID NOs: 2 (dao-1) and 3 (dao-2)) were amplified from genomic DNA by PCR using the Q5® High-Fidelity DNA polymerase, according to the manufacturer's instructions. The primers used for the amplification of dao-1 from the genomic DNA of Y. lipolytica PO1f were (5′-ATGACTCCCCACCCTTTCGATCAG-3); SEQ ID NO: 4) and (5′-CTAGATCTTGCAAGATCGACAGTCCTTG-3′; SEQ ID NO: 5). The primers for dao-1 were annealed at 69° C. for 30 s (35 cycles). The PCR product (2016 bp) was used directly for a second consecutive PCR amplification and restriction sites for BssHII and NheI were added. The primers used for this amplification were (5′-CTTGCGCGCATGACTCCCCACCCTTTC-3′; SEQ ID NO: 6) and (5′-GGCGCTAGCCTAGATCTTGCAAGATCG-3); SEQ ID NO: 7). The primers used for the amplification of dao-2 (2145 bp) from genomic DNA introduced restriction sites for BssHII (5′-GAAGCGCGCATGCACAGACTATCAC AACTAGC-3; SEQ ID NO: 8) and NheI (5′-CAAGCTAGCCTACTTGGAACAGC ACGA-3′; SEQ ID NO: 9) directly into the PCR product. The primers for dao-2 were annealed at 68° C. for 30 s (35 cycles). The amplicons dao-1_BssHII_NheI and dao-2_BssHII_NheI obtained were purified (DNA clean & concentrator-25, Zymo Research, Irvine, USA) before they were used for BssHII and NheI restriction digestion. The amplicons digested were purified using the GeneJET gel extraction kit (Thermo Fisher Scientific, Waltham, USA) after electrophoretic separation on a 1% (w/v) agarose gel. The genes were ligated (T4 DNA ligase) into a BssHII and NheI digested pHR_axp vector (FIG. 1), which comprises 1 kb flanking regions for the homologous recombinant integration into the acid extracellular peptidase (axp) locus of Y. lipolytica PO1f. Subsequently, chemically competent Escherichia coli XL-1 cells were transformed with the ligation approach by heat shock transformation. The cells were plated on lysogeny broth agar plates (100 μg/mL ampicillin) and grown overnight at 37° C. Colonies were picked and cultivated in 5 mL lysogeny broth media (100 μg/mL ampicillin) at 37° C. for 16 h and stirred at 180 rpm. The plasmids pHR_axp_dao-1 and pHR_axp_dao-2 were isolated using the GeneJET plasmid miniprep Kit (Thermo Fisher Scientific, Waltham, USA), according to the manufacturer's instructions. The dao genes were analyzed by sequencing (Eurofins Genomics GmbH, Germany) using primers for the axp flanking regions (5′-CTAAAGATGTTGATCTCCTTGTG CC-3′; SEQ ID NO: 10) and (5′-CCTCTGGGCCGAATACAACAC-3′; SEQ ID NO: 11) as known in the art.

Homologous Recombinant Integration of Dao Genes in Y. lipolytica PO1f

The genes for the putative DAOs dao-1 and dao-2 were homologously recombinantly integrated from the respective pHR vector (pHR_axp_dao-1 and pHR_axp_dao-2; FIG. 1) into the axp locus on the genome of Y. lipolytica PO1f using the CRISPR-Cas9 system, as known in the art.

Bioreactor Cultivation of Y. lipolytica PO1f_Axp_Dao-1

Preliminary experiments showed that DAO-2 did not show enzyme activity towards histamine but only towards agmatine. Therefore, DAO-2 was not further investigated in the present invention.

Y. lipolytica PO1f_axp_dao-1 and Y. lipolytica PO1f (negative control) were cultivated in the Multifors bioreactor system (1 L reactor volume, Infors HT) in a working volume of 800 mL. The bioreactors containing 720 mL of YPD_{Ura} media (YPD media+0.2 g/L uracil and 0.2 mL/L Antifoam 204) were inoculated with 80 mL preculture, which was cultivated in YPD_{Ura} in 500 ml shaking flasks and incubated at 30° C. for 20 h and stirred at 115 rpm. The bioreactors were constantly aerated with 0.5 vvm and heated to 28° C. The stirrer speed was stepwise increased (starting at 800 rpm) to keep the partial oxygen pressure pO₂ above 20%. The pH was kept constant at 6.5 using 2 M H₃PO₄ and 2 M NaOH. The pO₂ and pH values during the bioreactor cultivations were monitored using the InPro6900 and 405-DPAS-SC-K8S pH sensors, respectively. Samples were taken constantly over the course of the bioreactor cultivation to monitor the optical density (OD₆₀₀), bio dry mass and glucose concentration in the media. A 15 ml sample was taken for the determination of the intracellular DAO-1 activity every 8 h. These samples were centrifuged (8000 g, 7 min, 4° C.) and washed twice with saline (0.9% (w/v) NaCl), before the cell pellets were stored at −20° C. The entire cell mass was harvested after 96 h of cultivation by centrifugation (6000 g, 10 min, 4° C.). The pellet was washed twice with saline and finally centrifuged at 8000 g, 15 min, 4° C., before it was stored at −20° C. until further processed.

### Cell Disruption and Desalting for Protein Analysis and DAO Activity Determination

The yeast cells were thawed on ice and suspended (20% (w/v) cell suspension) in PIPES buffer (25 mM, pH 7.2) containing 0.1 mM PMSF (dissolved in 2-propanol). The enzyme samples taken over the course of the bioreactor cultivation were mechanically disrupted using a French press (SLM Aminco FA-078, American Instrument Exchange, Haverhill, USA) in two consecutive steps at 1 kbar. The cell mass harvested at the end of the cultivation was disrupted using a high-pressure homogenizer (One Shot 20 KPSI, Constant Systems Limited, Daventry, UK) in two consecutive steps at 1.35 kbar. The cell debris was removed by centrifugation (8000 g, 5 min, 4° C.) after cell disruption. The samples taken over the course of the bioreactor cultivation for the determination of enzyme activity were desalted after disruption using PD-10 columns (GE Healthcare, Chicago, USA) against PIPES buffer (25 mM, pH 7.2), according to the manufacturer's instructions.

### Purification of DAO-1

The DAO-1 was purified by fractionated (NH₄)₂SO₄ precipitation and subsequent hydrophobic interaction chromatography (HIC). Therefore, 10 g of Y. lipolytica PO1f_dao-1 cells were suspended and disrupted, as described above. Liquid (NH₄)₂SO₄ (4 M) was added dropwise (2 mL/min) to the cell-free extract to a final concentration of 20% (v/v) and further equilibrated for 1 h on ice with stirring (350 rpm). Thereafter, the solution was centrifuged at 10,000 g for 10 min at 4° C. The supernatant was precipitated again and the (NH₄)₂SO₄ concentration in the solution was increased from 20% (v/v) to 60% (v/v). The solution was centrifuged at 10,000 g for 10 min at 4° C. The pellet obtained was suspended in 45 mL sodium phosphate buffer (25 mM, pH 7) containing 1.3 M (NH₄)₂SO₄. The enzyme solution was filtered (0.45 μm) for the subsequent HIC purification using the resin Toyopearl Phenyl-650M (Column volume (CV)=22 mL). The sample was applied to the column at a flow rate of 2 mL/min. The column was washed with 4 CV of binding buffer (25 mM sodium phosphate buffer+1.3 M (NH₄)₂SO₄; pH 7.0) at 3 mL/min. The DAO-1 was eluted in a linear gradient of increasing (0 to 100% (v/v)) elution buffer (25 mM sodium phosphate buffer; pH 7.0) over 9 CV with a subsequent gradient delay of 50 mL. Samples taken during each purification step were desalted using PD-10 columns (GE Healthcare, Chicago, USA) against PIPES buffer (25 mM, pH 7.2), according to the manufacturer's instructions.

### Protein Analysis

The protein content of enzyme samples was determined according to Bradford, using BSA as a standard, as known in the art. Samples of the DAO purification procedure were analyzed by sodium dodecyl sulfate (SDS)-polyacrylamide gel electrophoresis (PAGE) on a 10% separating gel, as known in the art. An amount of 5 μg protein was loaded onto each lane of the SDS-PAGE. A protein molecular mass standard was used (Precision Plus Protein™ unstained protein standard 10-250 kDa) for molecular mass determination. Coomassie Brilliant Blue G-250 was used to stain the gel, as known in the art. The purified DAO-1 was additionally investigated by native PAGE analysis. The purified DAO-1 was applied twice on the native PAGE (each 5 μg protein), whereby one part was stained with Coomassie Brilliant Blue G-250 and the other part was active stained. The active staining solution contained 30 mM histamine, 50 UM DA-67 reagent, 5.32 U/mL horseradish peroxidase (Grade I) and 25 mM PIPES (pH 7.2). The active staining was continued at 37° C. until a blue band became visible.

### Dao Activity Determination

The DAO activity was determined using the colorimetric DA-67 enzyme assay, as known in the art. The reaction mixture, containing 750 μL histamine solution (30 mM; dissolved in 25 mM PIPES; pH 7.2; flushed with O₂ for 5 min) and 726 μL DA-67 reagent (10-(carboxymethyl-aminocarbonyl)-3,7-bis(dimethylamino) phenothiazine sodium salt; 50 μM; dissolved in 25 mM PIPES; pH 7.2) was incubated at 37° C. for 10 min and stirred at 750 rpm. Subsequently, 24 μL (266 units/mL) of horseradish peroxidase (Grade I) were added. The reaction was started by the addition of 50 μL enzyme solution and incubated at 37° C. for 10 min and stirred at 750 rpm. The reaction was stopped by the addition of 50 μL sodium diethyldithiocarbamate (30 mM). After centrifugation (10,000 g for 3 min at 20° C.), the absorption was measured at 668 nm. The histamine solution was replaced with buffer (25 mM PIPES; pH 7.2) for reference. Hydrogen peroxide (0.5 to 10 nmol/mL) was used for the calibration. The enzyme activity was calculated in nkat, whereby 1 nkat converts 1 nmol substrate/s at 37° C.

### Investigation of the Temperature and pH Profile of DAO-1

The influence of temperature on the enzyme activity of DAO-1 was tested under standard assay conditions, whereby the incubation temperature was varied between 2° and 47° C. The pH-dependency of the DAO-1 activity was investigated under standard assay conditions at 37° C., whereby different buffer systems (50 mM sodium phosphate buffer, pH 6.2-7.8; 50 mM PIPES, pH 7-7.5; 50 mM Tris-HCl, pH 7.2-8.5) were used. The respective buffer system was calibrated with hydrogen peroxide (0.5 to 10 nmol/mL). Analytically pure histamine was used as a substrate for the temperature and pH profiles.

Investigation of the pH-Stability of DAO-1

The influence of buffer and pH value on the stability of DAO-1 was investigated to determine suitable conditions for the histamine bioconversion experiment. Consequently, DAO-1 was incubated for 5 h at 37° C. and stirred at 400 rpm in the following buffer systems: 20 mM MES (pH 5.5), 20 mM sodium phosphate buffer (pH 6.2, 7.0, 7.2, 7.8, 8.0), 20 mM PIPES (pH 7.0, 7.2, 7.5) and 20 mM Tris-HCl (pH 6.8, 7.5, 8.2). Afterwards, the residual enzyme activity of DAO-1 was determined under standard assay conditions and compared to the DAO-1 activity applied initially.

### Kinetic Characterization of DAO-1

The apparent kinetic parameters of DAO-1 were determined by Michaelis-Menten kinetics with histamine as the substrate under standard assay conditions. The histamine concentration (analytically pure histamine) was varied between 0.5 and 50 mM. Kinetic investigations were done within the initial reaction velocity.

### Investigation of the Substrate Selectivity of DAO-1

The substrate selectivity of DAO-1 was tested with different food industry-relevant biogenic amines as substrates (tryptamine, cadaverine, putrescine, agmatine, spermidine, histamine, and tyramine) under standard assay conditions. The amino acids L-tryptophan, L-lysine, L-histidine, and L-arginine were also tested. Each substrate was used at concentrations of 1, 10 and 50 mM in the final assay approach, except tryptamine, which was tested only at 1 mM due to its insolubility at higher concentrations.

Histamine Bioconversion with DAO-1

The histamine reduction experiments were done, as known in the art, with a food-relevant histamine concentration of 150 mg/L histamine. The histamine reduction was done at 37° C. in PIPES buffer (20 mM, pH 7) for 5 h. An amount of 5 g/L BSA and 25 g/L sucrose were added to the approach to simulate a food matrix. Additionally, catalase was added (30 nkat/mL) to remove the hydrogen peroxide. The experiment was started by the addition of 0.1 nkat/mL DAO-1 activity. This DAO activity was determined under modified assay conditions, whereby the buffer system (20 mM PIPES, pH 7), substrate concentration (1.35 mM histamine in the final assay approach) and additives (5 g/L BSA and 25 g/L sucrose) were adjusted to the bioconversion conditions. The histamine reductions were determined by reversed-phase high-performance liquid chromatography with thiamine (1.35 mM) as an internal standard, as known in the art.

### Statistical Analysis

All experiments, except the DAO-1 production in the bioreactor, were carried out at least in duplicate and evaluated by determining the standard deviation with Excel (Microsoft, Redmond, USA). Data are presented as mean values with standard deviation. Enzyme kinetics were evaluated by nonlinear regression using the data analyzing software Sigmaplot 12.5 (Systat Software GmbH, Erkrath, Germany).

### Example 1

Identification of Dao Genes in Y. lipolytica PO1f and their Expression in Shaking Flask Experiments

Y. lipolytica PO1f was identified as the owner of two putative DAOs using the BLAST program (https://blast.ncbi.nlm.nih.gov). Both genes did not contain any introns. An amino acid sequence comparison of porcine DAO (Uniprot: Q9TRC7) with the two putative DAOs showed a query cover of 54% and an identity of 25% for DAO-1 and a query cover of 53% and an identity of 25% for DAO-2 (FIG. 2A, B). Comparing both putative DAOs with each other showed a query cover of 97% and an identity of 35%. Further, an amino acid sequence comparison of porcine and human (Uniprot: P19801) DAO with the two putative DAOs showed a consensus of around 21% for both DAO-1 and DAO-2 (FIGS. 13, 14). Comparing both putative DAOs with each other showed a similarity of around 34%. Both genes were amplified from the genome of the Y. lipolytica strain PO1f and successfully integrated into the axp locus of the genome (integration efficiency of around 20%), resulting in the recombinant strains Y. lipolytica PO1f_axp_dao-1 and Y. lipolytica PO1f_axp_dao-2. The genes were integrated into the axp locus because it allowed efficient expression of the proteins desired with the chosen strategy for homologous recombinant integration, as known in the art. Gene expression was thereby conducted using the strong, constitutive promoter UAS1B8_TEF (136). In comparison to the wildtype Y. lipolytica PO1f cell extract, the cell extract of Y. lipolytica PO1f_axp_dao-2 did not show an increase in DAO-activity for the substrates histamine, putrescine, cadaverine, tyramine, spermidine, and tryptamine. Only for the substrate agmatine a 5.5-fold increase in activity was measured (around 50 nkat_{Agmatine} (14.5 mM)/L_{Culture}). Since no overexpression of DAO-2 in the recombinant Y. lipolytica strain was visible on SDS-PAGE (data not shown) and the enzyme showed no activity towards histamine, only DAO-1 was investigated further in this study.

### Example 2

Production of DAO-1 in Y. lipolytica PO1f

Y. lipolytica PO1f_axp_dao-1 was cultivated in the Multifors bioreactor system in working volumes of 800 mL in YPD medium supplemented with 0.2 g/L uracil over 96 h. The original Y. lipolytica PO1f strain was cultivated identically in parallel as a reference. Both strains showed similar growth behaviors reaching a maximal bio dry mass of around 24 g/L after 16 h as shown in FIG. 3. This corresponded to a maximal optical density of around 60 (maximal specific growth rates μ of 0.32 h⁻¹). The bio dry mass apparently decreased to around 15 g/L after 96 h for both strains, which was attributed to inhomogeneous sample taking over the course of the bioreactor cultivation. The volumetric DAO-1 activity (intracellular) of the Y. lipolytica PO1f_axp_dao-1 strain increased linearly over the course of the bioreactor cultivation, reaching a maximum of 2784±75 nkat/L_{Culture} after 56 h. Then, the DAO-1 activity decreased slightly, reaching a final volumetric DAO-1 activity of 2343±98 nkat/L_{Culture} with a specific DAO-1 activity of 1301±54.2 nkat/g_{Protein}. The reference Y. lipolytica PO1f had a maximal volumetric DAO activity of 58±0.6 nkat/L_{Culture} with a specific DAO activity of 14.1±0.14 nkat/g_{Protein} after 48 h of cultivation. It is suggested that the DAO activity of the reference strain Y. lipolytica PO1f is due to the native DAO-1 expression, since DAO-2 showed no activity towards the substrate histamine, which was used for the determination of DAO activity. Therefore, the homologous recombinant integration of the dao-1 gene into the axp locus of Y. lipolytica PO1f allowed efficient expression of DAO-1.

The specific DAO-1 activity of 1301±54.2 nkat/g_{Protein} obtained in the crude extract was around 16 times higher than the DAO activity of the partially purified porcine DAO known in the art with 81 nkat/g_{Protein}. If DAO is used as a dietary supplement, a considerably high activity of DAO must be used to degrade the food-relevant histamine amounts. The enzyme activity of porcine DAO required for the degradation of 75 mg histamine was 50 nkat within 5 h under in vitro conditions. To obtain this DAO activity, 100 g of pig kidney were needed for sufficient DAO extraction. By comparison, the isolation and partial purification of DAO from almost kg pig kidneys would result in the same amount of DAO-1 activity obtained from one liter of bioreactor cultivation of Y. lipolytica PO1f_axp_dao-1. Therefore, the microbial DAO production seems cost-effective and offers several advantages compared to the use of DAO from pig kidney, especially in terms of ethical and cultural considerations.

### Example 3

Purification of DAO-1 from Crude Extract after Cell Disruption

The yeast DAO-1 was purified by fractionated (NH₄)₂SO₄ precipitation and subsequent HIC. From 10 g of wet yeast biomass, 108±5 nkat of DAO-1 activity (specific DAO-1 activity of 660±28 nkat/g_{Protein}) was obtained after cell disruption using a high-pressure homogenizer. After (NH₄)₂SO₄ precipitation and HIC (FIG. 4), the DAO-1 was purified 7.2-fold (specific enzyme activity of 4738±31 nkat/g_{Protein}) with a yield of 46.6±0.31%. By comparison, the purification of DAO from porcine kidney yielded a specific enzyme activity of 81±1 nkat/g_{Protein} with a similar purification strategy employing a heat treatment, salt precipitation and HIC. Thus, the production of a DAO in a microbial production host and the subsequent purification achieved much higher specific DAO activities. The purification of DAO-1 was evaluated by SDS-PAGE analysis (FIG. 5) showing a protein band with increasing intensity over the purification procedure at around 75 kDa, which was in accordance with the theoretical monomeric molecular weight of DAO-1 (75.4 kDa as calculated from amino acid sequence). The SDS-PAGE also showed that the DAO-1 preparation still contained other proteins. However, it was shown on an active-stained native PAGE (FIG. 6) that only one protein band at around 146 kDa was active towards the substrate histamine. This indicated that the purity of the DAO-1 preparation was sufficient for further biochemical characterization. The DAO-1 seemed to be a homodimeric enzyme based on the size observed in SDS-PAGE and native PAGE analysis, respectively. This was in accordance with the homodimeric structure observed for most of the copper amine oxidases including human and porcine DAO described in the art.

### Example 4

### Biochemical Investigation of DAO-1

The purified DAO-1 was investigated regarding the influence of temperature, pH value and buffer on the DAO-1 activity. The DAO-1 had the maximal activity at 40° C. (FIG. 7). An increase of the temperature to 42 or 45° C. decreased the relative DAO-1 activity to 76±1% and 31.7±1%, respectively. The relative DAO-1 activity was greater than 50% for a temperature range between 3° and 42° C. Therefore, the temperature profile of DAO-1 was comparable to the temperature profiles of other DAOs, for example, that from pig and human, however, these DAO activities were determined with putrescine as the substrate. The DAO-1 showed the maximal DAO activity at pH 7.2 in Tris-HCl buffer (50 mM) (FIG. 8). Due to the limited buffering capacity of Tris-HCl at this pH value, the following experiments were done in PIPES buffer, which resulted in a slightly lower DAO-1 activity of 92.2±3.9% compared to the maximal activity in Tris-HCl at pH 7.2. The pH-profile also seems to be comparable to human and porcine DAOs. The prior art found that the maximal DAO activity of human DAO (from blood serum) with putrescine as the substrate was at pH 7.5 in phosphate buffer. The DAO from pig kidney showed the maximal DAO activity towards histamine and cadaverine in potassium phosphate buffer at pH values of 6.3 and 7.4, respectively. The highest stability of DAO-1 was determined in PIPES buffer (20 mM) at pH 7.0 with a residual DAO-1 activity of 89.5±0.8% after incubation at 37° C. for 5 h (FIG. 9). The DAO-1 still retained 53.7±1.2% of its activity (data not shown) after 24 h at 37° C. The DAO-1 maintained more than 60% of 390 its activity after a 5 h incubation at 37° C. in a pH range from 6.2 to 7.5.

### Example 5

### Kinetic Investigation of DAO-1

The kinetic investigation of DAO-1 was carried out with histamine concentrations ranging from 0.5 to 50 mM (FIG. 10). The linearization of the Michaelis-Menten curve, according to Hanes-Woolf, is shown in FIG. 11. A substrate inhibition by histamine was recognized for histamine concentrations greater than 12.5 mM. The Kₘ value of DAO-1 for histamine was 2.3±0.2 mM (R²=0.985) with a Vₘₐₓ of 9.09±0.37 nkat/mg. Furthermore, a Kᵢ of 61.89±8.51 mM was determined. Therefore, the affinity of DAO-1 to the substrate histamine is less compared to mammalian DAOs from porcine or human, which have Kₘ values for histamine of 0.027 and 0.0028 mM, respectively. However, microbial amine oxidases seem to generally have less affinity towards histamine compared to mammalian DAOs. Kₘ values of 0.5 and 0.6 mM were determined in the art for a histamine oxidase from Arthrobacter crystallopoietes KAIT-B-007 and an amine oxidase from Aspergillus niger, respectively, with histamine as substrate. The comparatively lower affinity of microbial DAOs towards histamine might derive from the fact that mammalian DAOs are detoxifying enzymes that must be able to degrade biogenic amines at very low concentrations with high efficiency. However, the DAO in microorganisms seems to be primarily important for the nitrogen metabolism and, therefore, does not need to have a very high affinity towards histamine.

The porcine and human DAOs are also substrate inhibited by histamine but at distinctively lower concentrations of Kᵢ=5.71 and 0.28 mM, respectively. Therefore, DAO-1 degrades histamine at higher concentrations with a higher reaction rate.

### Example 6

### Investigation of the Substrate Selectivity of DAO-1

The DAO-1 showed a broad substrate selectivity with different food-relevant biogenic amines as substrates (Tab. 1). The highest DAO-1 activity was determined with 1 mM tyramine as the substrate. The DAO-1 seemed to be substrate-inhibited by tyramine, as an increase of the substrate concentration caused a reduction of the DAO-1 activity. The same inhibitory effect was also observed for the other substrates: Histamine, putrescine, cadaverine, agmatine and spermidine. The third highest DAO-1 activity was measured with histamine as the substrate at 10 mM, which was set as a reference (100%) since it was the biogenic amine with the highest relevance for the present invention. The DAO-1 did not show any activity towards the amino acids L-tryptophan, L-lysine, L-histidine, and L-arginine tested.

TABLE 1
Substrate selectivity of DAO-1 towards food-relevant
biogenic amines at 1, 10 and 50 mM. DAO-1 activity measured
with the DA-67 assay in PIPES buffer (25 mM; pH 7.2).
100% (10 mM histamine) = 1.45 ± 0.01 nkat/mL.

| | DAO-1 activity [%] | | |
|---|---|---|---|
| Substrate | 1 mM | 10 mM | 50 mM |
| Histamine | 45.1 ± 0.7 | 100 ± 1.5 | 74.4 ± 1.1 |
| Tyramine | 303.9 ± 4.4 | 246.8 ± 3.6 | 66.4 ± 1.0 |
| Putrescine | 29.8 ± 0.4 | 92.9 ± 1.4 | 77.9 ± 1.1 |
| Cadaverine | 29.6 ± 0.4 | 87.3 ± 1.3 | 64.5 ± 1.0 |
| Agmatine | 31 ± 1.1 | 30.6 ± 2.7 | 7.4 ± 2.3 |
| Spermidine | 2.1 ± 1.6 | 3.8 ± 0.9 | 2.1 ± 1.3 |
| Tryptamine | 166.1 ± 0.8 | not soluble | not soluble |

The amino acids L-tryptophan, L-lysine, L-histidine, and L-arginine were tested but did not show DAO-1 activity.

The biogenic amines tyramine, histamine, putrescine and cadaverine are especially of high relevance when it comes to biogenic amine-induced toxicity from foods. The DAO-1 was capable of degrading all of these relevant biogenic amines with high efficiency. This broad substrate selectivity for food-relevant biogenic amines seems to be a rarely found feature of microbial amine oxidases. The histamine oxidase from Arthrobacter crystallopoietes KAIT-B-007 and an amine oxidase from Aspergillus niger, for example, showed no activity towards the substrates putrescine, cadaverine, spermine and spermidine but showed activity towards histamine. A phenylethylamine oxidase from Arthrobacter globiformis was also found in the art to be active towards tyramine but was poorly active towards histamine and not active towards putrescine.

The mammalian DAO from porcine kidney showed the highest activity towards the aliphatic diamines putrescine and cadaverine in the art, both at 1 mM. Here, the DAO activity measured with histamine was 19% compared to the maximal measured activity with putrescine.

### Example 7

Histamine Bioconversion with DAO-1

The DAO-1 was used in histamine bioconversion experiments to evaluate its efficacy for the reduction of histamine in food-relevant concentrations (150 mg/L; 1.35 mM), as known in the art, with modifications regarding the buffer system (20 mM PIPES; pH 7) and used additives to simulate a food matrix (5 g/L BSA, 25 g/L sucrose, 30 nkat/mL catalase) (FIG. 12). The DAO-1 preparation reduced the histamine by 74.9±1.73% to 37.4±0.65 mg/L (0.34±0.01 mM) using 0.1 nkat/mL within 5 h at 37° C. Although the histamine bioconversion experiments were carried out at histamine concentrations slightly above the Kₘ value of DAO-1 (2.3±0.2 mM), histamine was steadily reduced until a stagnation was recognized, starting at around 4 h at a histamine concentration of 43.5±0.76 mg/L (0.39±0.01 mM). The residual activity of DAO-1 after this 5 h bioconversion was 57.4±1.3% (determined by RP-HPLC). Therefore, the stagnation was attributed to the enzyme kinetics of DAO-1, which decelerated the enzyme catalysis at lowered histamine concentrations. Nevertheless, the DAO-1 kinetics were sufficient for the conversion of around 75% of the histamine applied. Around 90% of the histamine applied (150 mg/L) were reduced using the same enzyme activity as used herein in previous studies with porcine DAO. Therefore, porcine DAO was also unable to completely degrade the histamine in the experimental setup, which was attributed to a potentially product inhibitory effect by (imidazol-4-yl) acetaldehyde. However, it was also stated that this effect is difficult to assess for the histamine conversion in actual foods, because the reaction product might react further to other compounds and, therefore, would not disturb the actual histamine conversion.

### Example 8

### A DAO Tablet for the Treatment of Histamine Intolerance by Oral Supplementation

### Abstract:

DAO-1 from Y. lipolytica was investigated for its histamine degradation capability under simulated intestinal conditions (SIF). Therefore, DAO-1 was formulated together with catalase as a sucrose-based tablet. The tablet (9×7 mm; 400 mg) contained 690 nkat of DAO-1 activity, which were obtained from a bioreactor cultivation of a genetically modified Y. lipolytica PO1f with optimized downstream-processing. The DAO-1 tablet was tested in a histamine bioconversion experiment under SIF conditions with the addition of a simulated food matrix, whereby 22 mg of the initially applied 75 mg histamine were degraded. This amount could already be sufficient to circumvent symptoms of a histamine intolerance. Furthermore, it was found that the stability of DAO-1 in SIF is distinctively influenced by the presence of a food-matrix, indicating that the amount and type of food consumed can affect the oral supplementation with DAO. This study showed for the first time that a microbial DAO could have the potential for the treatment of histamine intolerance by oral supplementation.

### Introduction

According to the European Food and Safety Authority (EFSA) and the European Centre for Disease Prevention and Control (ECDC) the biogenic amine histamine is associated with increasing numbers of foodborne illness outbreaks in the European Union. Thereby, foods containing histamine levels of 500 mg/kg and above can be considered as hazardous for the human health. However, also the consumption of foods with moderate or even low histamine concentrations can negatively affect humans who suffer from a histamine intolerance. This seems to apply for around 1% of the total population. Typical symptoms of this condition can be gastrointestinal disorders, headaches, asthma, flushing and sneezing. The intolerance towards minor dosages of exogenous histamine results from an imbalance between the uptake of histamine and the histamine degrading enzyme DAO. DAO is a secretory enzyme that is mainly located in the small intestinal mucosa but can also be found in the circulation. It catalyzes the oxidative deamination of histamine or other biogenic amines to the corresponding aldehydes, ammonia, and hydrogen peroxide. Several factors can affect the available activity of DAO in humans. First, single-nucleotide polymorphisms in the DNA sequence of DAO can decrease the productivity or kinetics of DAO, lowering the serum DAO activity. The serum DAO activity has been found to be significantly lower in patients suffering from symptoms of a histamine intolerance, suggesting that it can be used as a diagnostic tool for histamine intolerance. Additionally, it was shown that the serum DAO activity seems to be in direct correlation with the status of the intestinal mucosa. A reduced activity can therefore be observed for various gastrointestinal disorders and injuries. However, this can be a temporary effect as it was shown in patients undergoing chemotherapy that they were able to recover from decreased serum DAO activities within a few weeks. Furthermore, the available DAO activity is affected by the intake of other biogenic amines, drugs, or alcohol. Since exogenous histamine enters the body primarily in the small intestine and can surpass into the circulation through the intestinal wall, an efficient degradation by DAO on-site is of high relevance. Therefore, a solution approach could be the supplementation of DAO to support the insufficient endogenous DAO in the small intestine. Commercially available dietary supplements that contain DAO from a pig kidney extract were investigated for the potential in the treatment of histamine intolerance in several clinical studies. Thereby, it was found to be effective in the treatment of histamine intolerance-associated symptoms. However, the preparation was recently tested in an in vitro histamine bioconversion experiment, whereby no DAO activity was determinable. Furthermore, it was shown that considerable high DAO activities (50 nkat) must be supplemented to degrade food-relevant histamine amounts. Therefore, it might be difficult to provide this level of DAO activity by extraction from natural sources such as pig kidney. As an alternative approach, the overexpression in microbial hosts and subsequent downstream processing might result in a much higher DAO productivity, suitable for the production of highly efficient DAO tablets for oral supplementation. DAO-1 from Y. lipolytica as described herein showed promising characteristics for the administration in the food industry or as a dietary supplement as it was able to degrade several food-relevant biogenic amines (tyramine, putrescine, cadaverine, histamine).

The aim of this study was to investigate the potential of the newly discovered DAO-1 from Y. lipolytica for the treatment of histamine intolerance as oral supplement. Therefore, DAO-1 was formulated as a tablet and applied for the conversion of food-relevant histamine amounts under simulated intestinal conditions. Furthermore, stability and kinetics of DAO-1 under these conditions were assessed.

### Material and Methods

### Materials and Reagents:

1,4-piperazinediethanesulfonic acid (PIPES), histamine dihydrochloride, sodium hydroxide (NaOH), D(+)-sucrose, monobasic potassium phosphate (KH₂PO₄), hydrochloric acid (HCl) and hydrogen peroxide (30%) were purchased from Carl Roth GmbH (Karlsruhe, Germany). Sodium dihydrogen phosphate, sodium diethyldithiocarbamate, ortho-phosphoric acid (H₃PO₄) and thiamine chloride dihydrochloride were purchased from Merck (Darmstadt, Germany). Bovine serum albumin (BSA; modified Cohn Fraction V, pH 5.2) was purchased from Serva electrophoresis GmbH (Heidelberg, Germany). Catalase (from Micrococcus lysodeikticus; 111700 U/mL) and pancreatin from porcine pancreas (8×USP specifications) was purchased from Sigma-Aldrich (Merck) (St. Louis, USA). (10-(carboxymethyl-aminocarbonyl)-3,7-bis(dimethylamino) phenothiazine sodium salt (DA-67) was purchased from Fujifilm Wako Chemicals U.S.A. Corp (Richmond, USA). Horseradish peroxidase (Grade I) was purchased from AppliChem GmbH (Darmstadt, Germany). Whey protein isolate (90% (w/w) protein) was obtained from Sachsenmilch Leppersdorf GmbH (Leppersdorf, Germany). Sodium-caseinate (90.6% (w/w) protein) was obtained from FrieslandCampina (Amersfoort, Netherlands).

### Production and Purification of DAO-1:

The production of DAO-1 was done using a genetically modified Y. lipolytica PO1f strain (Y. lipolytica PO1f_axp_dao-1). Here, the native DAO-1 gene was integrated into the axp locus on the genome of Y. lipolytica using the CRISPR-cas9 system. The DAO-1 expression was conducted using the strong and constitutive UAS1B8-TEF (136) promotor. Y. lipolytica PO1f_axp_dao-1 was cultivated in the Labfors 5 bioreactor system (Infors GmbH, Einsbach Germany) in a working volume of 5 L. Cells were harvested after 56 h of cultivation and were stored at −20° C. until they were disrupted. For disruption, 150 g cells were thawed on ice and used to prepare a 20% (w/w) suspension in PIPES buffer (25 mM, pH 7). Cell disruption was done in a stirred media mill (Dyno®-Mill Typ KDL A; Willy A. Bachofen AG Maschinenfabrik, Muttenz, Swiss) at 2500 rpm using glass beads with a diameter of 0.75 mm. The system was cooled to 5° C. using an Ultra-Kryomat® RUK50 (Lauda Dr. R. Wobser GmbH & Co. KG, Lauda-Königshofen, Germany). The cell suspension was continuously fed to the Dyno®-Mill system with a peristaltic pump with a rate of around 14 mL/min, providing a residence time of 18 minutes. Afterwards, the glass beads were washed with 750 mL PIPES buffer (25 mM, pH 7) at around 14 mL/min with the stirred media mill still running at 2500 rpm. The initial cell lysate as well as the buffer used for washing the glass beads were pooled and centrifuged (8,000 g, 4° C., 10 min). Around 800 mL of supernatant were collected and further purified by an ammonium sulfate precipitation (60% (v/v) 4 M (NH₄)₂SO₄) and hydrophobic interaction chromatography as known in the art. The purified DAO-1 was stored at −80° C.

### Dao-1 Activity Determination:

The DAO-1 activity was determined using a colorimetric DA-67 enzyme assay. The reaction mixture, containing 375 μL histamine solution (30 mM; dissolved in 25 mM PIPES; pH 7.2) and 363 μL DA-67 reagent (10-(carboxymethyl-aminocarbonyl)-3,7-bis(dimethylamino) phenothiazine sodium salt; 50 M; dissolved in 25 mM PIPES; pH 7.2) was incubated at 37° C. for 10 min and stirred at 750 rpm. Subsequently, 12 μL (266 units/mL) of horseradish peroxidase (Grade I) were added. The reaction was started by the addition of 25 μL DAO solution and incubated at 37° C. for 10 min and stirred at 750 rpm. The reaction was stopped by the addition of 50 μL sodium diethyldithiocarbamate (30 mM). After centrifugation (10,000 g for 3 min at 20° C.), the absorption was measured at 668 nm. The histamine solution was replaced with buffer (25 mM PIPES; pH 7.2) for reference. Hydrogen peroxide (0.5 to 10 nmol/mL) was used for the calibration. The enzyme activity was calculated in nkat, whereby 1 nkat converts 1 nmol substrate/s at 37° C.

### Protein Analysis:

The protein content of the DAO-1 preparation was determined according to Bradford, using BSA as a standard. Additionally, the DAO-1 preparation used for the preparation of the tablets was investigated by sodium dodecyl sulfate (SDS)-polyacrylamide gel electrophoresis (PAGE) on a 10% separating gel. An amount of 5 μg protein was loaded onto the SDS-PAGE. A protein molecular mass standard was used (Precision Plus Protein™ unstained protein standard 10-250 kDa) for molecular mass determination. Coomassie Brilliant Blue G-250 was used to stain the gel.

### Stability of DAO-1 in Simulated Intestinal Fluid:

The stability of DAO-1 was tested in a simulated intestinal fluid (SIF) with and without the addition of simulated food matrices. Therefore, pancreatin-containing SIF was prepared as described in the United States Pharmacopeia (USP42). Different food-matrix stock solutions (4× concentrated) were prepared as follows: 200 g/L bovine serum albumin (BSA) and 100 g/L sucrose (=food-matrix 1), 100 g/L BSA, 100 g/L whey protein isolate (WPI) and 200 g/L sucrose (=food-matrix 2) and BSA, WPI, sodium-caseinate each at 66.68 g/L and 200 g/L sucrose (=food-matrix 3). The pH of each stock solution was adjusted to 6.8 with 1 M NaOH. The freshly prepared 2× concentrated SIF, the food-matrix stock solutions as well as DAO-1 (desalted against H₂O_{dd} using PD MidiTrap G-25 columns; GE Healthcare, Chicago, USA) were individually incubated for 5 min in a thermoshaker at 37° C. Subsequently, 500 μL of 2× concentrated SIF, 250 μL of DAO-1 and 250 μL of the food-matrix stock solutions were combined and incubated at 37° C., 500 rpm in a thermoshaker. In order to test the stability of DAO-1 without the presence of stabilizing compounds, H₂O_{dd} was added instead of the food-matrix stock solution. Immediately after combining the solutions, a sample of 100 μL was taken and used for the DAO-1 activity determination using the DA-67 assay.

### Kinetics of DAO-1 in a Simulated Intestinal Fluid:

The apparent kinetic parameters of DAO-1 were determined by Michaelis-Menten kinetics with histamine as the substrate (1.56 to 50 mM) in a food-matrix containing SIF under unhydrolyzed conditions (without pancreatin) and under hydrolyzed conditions. The DAO-1 activity was determined using the DA-67 assay. For unhydrolyzed conditions, the DA-67 reagent (50 μM) was prepared in SIF containing BSA, WPI, sodium-caseinate each at 35.6 g/L and 106.8 g/L sucrose (pH 6.8). The histamine and horseradish peroxidase were dissolved in SIF. For histamine, the pH was readjusted to 6.8 using 1 M NaOH. The DAO-1 was desalted against SIF using PD MidiTrap G-25 columns.

For the hydrolyzed conditions, BSA, WPI, sodium-caseinate each at 16.67 g/L and 50 g/L sucrose were first incubated in SIF (with pancreatin) at 37° C. and 130 rpm for 90 min. The hydrolysis was stopped by heating the solution at 95° C. for 15 min. Subsequently, it was centrifuged (8,000 g, 4° C., 10 min). The supernatant was then used to dissolve the DA-67 reagent (50 μM), histamine, horseradish peroxidase, and to dilute the DAO-1. The pH of the histamine solution was readjusted to 6.8 using 1 M NaOH. For the calibration, histamine was replaced by hydrogen peroxide (0.5 to 20 nmol/mL). Kinetic investigations were done within the initial reaction velocity.

### Preparation of DAO-1 Tablets:

The purified DAO-1 was first concentrated by ammonium sulfate precipitation. Therefore, liquid ammonium sulfate (4 M) was added dropwise under stirring and on ice to 335 mL purified DAO-1 solution to a final concentration of 60% (v/v). After completing the addition of liquid ammonium sulfate, the approach was further incubated for 60 min on ice. Then, it was centrifuged (8,000 g, 4° C., 25 min). The supernatant was completely removed, and the pellet was dissolved in 3 mL sodium phosphate buffer (20 mM, pH 7). Sucrose powder was added to a final concentration of 40 g/L. Furthermore, 60 μkat of catalase from Micrococcus lysodeikticus were added. The final DAO-1 solution was divided in 4 parts which were separated in weighed 2 mL Eppendorf reaction tubes. These were frozen at −80° C. before they were freeze-dried. The freeze-dried powders were mixed with sucrose at a ratio of 50/50 (w/w) before they were used to prepare the DAO-1 tablets with a self-built tablet press (FIG. 15).

### Histamine Bioconversion in a Food-Matrix SIF Using DAO-1 Tablets:

A histamine bioconversion was done using the DAO-1 tablets in a food-relevant histamine concentration of 1.35 mM (150 mg/L) as known in the art. The experiment was done in a 500 mL approach volume in 1 L Erlenmeyer flasks that contained the food-matrix SIF 3 (BSA, WPI, sodium-caseinate at 16.67 g/L and sucrose at 50 g/L in SIF (pH 6.8)) and 75 mg histamine. The bioconversion was done in triplicate. The approaches were preincubated at 37° C. for 2 h. Pancreatin (20 g/L in SIF) was preincubated for 5 min at 37° C. and was added to a final concentration of 1.25 g/L. Immediately after mixing the approaches, samples of 2 mL were taken, which were inactivated at 95° C. for 5 min in a water bath and then treated as described herein. Furthermore, a 20 ml sample was taken from a reference bioconversion approach (without histamine), which was cooled down in an ice-water bath for the subsequent preparation of a histamine calibration for the reversed-phase high-performance liquid chromatography (RP-HPLC) analysis of the initial histamine concentration. Therefore, a histamine stock solution was diluted in the reference approach media to histamine concentrations between 0.25 and 2 mM. These calibration samples were heated at 95° C. for 5 min in a water bath and then treated as described below. The histamine bioconversion was started by the addition of one DAO-1 capsule to each approach. Additionally, a DAO-1 tablet was also added to the reference approach. No DAO-1 capsule was added to a negative control approach. The flasks were incubated on a rotary shaker at 37° C. and 130 rpm for 90 min. Samples of 2 mL were taken after 30, 50, 70 and 90 min and were inactivated at 95° C. for 5 min in a water bath and treated as described below. After 90 min, a sample of 20 mL was taken from the reference approach and was cooled down in an ice-water bath. Subsequently, histamine calibration samples (0.1 to 1.5 mM) for the RP-HPLC analysis of the histamine concentration in the bioconversion samples (30-90 min) were prepared as described above.

### Sample Preparation of Bioconversion Samples for the RP-HPLC Analysis:

Heat-inactivated samples from the histamine bioconversion were cooled down on ice before they were centrifuged (10,000 g, 4° C., 3 min). The supernatant (1 mL) was loaded on a PD MidiTrap G-25 column which was equilibrated with H₂O_{dd}. Undigested proteins and large peptides were eluted from the column using 1.5 mL H₂O_{dd} and discarded. Histamine and molecules of low molecular weight were eluted in 2 mL H₂O_{dd} and collected. The pH-value of these samples was adjusted to around 2 using 35 μL HCl (1 M). The samples were kept at 20° C. in a thermoshaker until they were further purified.

The cation exchange material Lewatit®S100 (275 mg) was filled in a 1 mL pipette tip, which was loosely sealed at the bottom and top with cotton wool. The material was then washed with 4 mL of H₂O_{dd}. Afterwards, it was equilibrated with 4 mL of HCl (10 mM). The pH-adjusted bioconversion samples were then applied (1 mL each) to the cation exchange material. The material was washed with 5 ml of H₂O_{dd}. Then, 600 μL of ammonia (4 M) were added to the material, which were discarded. Again 600 μL of ammonia (4 M) were added to elute histamine. The ammonia water was evaporated at 70° C. and 500 rpm in a thermoshaker overnight. The remains were dissolved in 200 μL HCl (10 mM). Then, 50 μL of internal standard solution (thiamine chloride dihydrochloride; 6 mM in H₂O_{dd}) were added for the RP-HPLC analysis. The pH of each sample was adjusted to around 2 by addition of 5 μL HCl (1 M). Samples were centrifuged (20,000 g, 4° C., 5 min) before they were analyzed by RP-HPLC.

### RP-HPLC Analysis of Histamine in Bioconversion Samples:

The histamine concentration in bioconversion samples was determined by RP-HPLC as known in the art. The mobile phase consisted of 92.5% (v/v) 20 mM NaH₂PO₄ and 10 mM octane-1-sulfonic acid sodium salt (pH adjusted to 2.2 using 4 M H₃PO₄) and 7.5% (v/v) acetonitrile. The injection volume was set to 5 μL. The separation was done at 40° C. at a constant flow rate of 1 mL/min for 25 min.

### Statistical Analysis:

All experiments were done at least in duplicate and evaluated by determining the standard deviation with Excel. Data are presented as mean values with standard deviation. Enzyme kinetics were evaluated by nonlinear regression using the data analyzing software Sigmaplot 12.5 (Systat Software GmbH, Erkrath, Germany).

### Results and Discussion:

### Larger-Scaled Production of DAO-1 for the Preparation of DAO-1 Tablets:

In a previous study it could be shown that DAO-containing tablets with high DAO activities (50 nkat) might be required to treat histamine intolerance by oral supplementation. Therefore, DAO-1 from Y. lipolytica, as described herein, was recombinantly produced in the yeast Y. lipolytica PO1f in a bioreactor with a working volume of 800 mL, as known in the art. The harvested cell mass was disrupted in a smaller scaled high-pressure homogenizer yielding 10.8 nkat DAO-1 activity per gram wet yeast cells equaling around 800 nkat DAO-1 activity in total. However, this activity is not sufficient for the preparation of highly active DAO-1 tablets. Therefore, the bioreactor cultivation was repeated in a 5 L scale, whereby an optical density (OD₆₀₀) of 53, bio dry mass of 22 g/L and wet yeast mass of 93 g/L were reached after 56 h of cultivation (FIG. 16). This was followed by a larger-scaled cell disruption using a stirred media mill. Thereby, 150 g of wet yeast cells were disrupted, yielding around 4.8 μkat of DAO-1 activity. This equaled a yield of 32 nkat per gram wet cells which is around 3 times higher than in the previous study. The DAO-1 was purified by ammonium sulfate precipitation and hydrophobic interaction chromatography, which yielded a total of 3 μkat with a specific DAO-1 activity of 15 μkat/g_{Protein}. Therefore, also the specific DAO-1 activity was increased 3-fold as compared to the recent work (4.7 nkat/g_{Protein}). This increased specific DAO-1 activity is crucial to provide a highly active DAO-1 extract in the limited capacity of a tablet.

### Stability of DAO-1 in a Simulated Intestinal Fluid (SIF):

Since DAO-1 will be applied to degrade histamine in the human intestine, DAO-1 stability under these conditions is of high relevance. The intestinal environment can be imitated with a simulated intestinal fluid (SIF) as described in the United States Pharmacopeia (USP42). This SIF contains pancreatin, which is an enzyme preparation with different enzyme activities like amylases, peptidases, and lipases. The purified DAO-1 was tested in this SIF with and without the presence of simulated food-matrices at 37° C. (FIG. 17). Thereby, DAO-1 showed weak stability in pure SIF with a half-life period of less than 5 min. The addition of 50 g/L BSA and 25 g/L sucrose (food-matrix SIF 1) improved the stability of DAO to a half-life period of around 15 min. This improvement in stability was most likely due to the increased number of substrates being available for the pancreatic peptidases. The addition of 25 g/L BSA, 25 g/L WPI and 50 g/L sucrose (food-matrix SIF 2) further improved the DAO-1 stability to a half-life period of around 30 min. Maintaining the total sucrose and protein concentration at 50 g/L but introducing sodium-caseinate as a third protein (food-matrix SIF 3; BSA, WPI and sodium-caseinate each at 16.67 g/L and 50 g/L sucrose) further improved the long-term stability of DAO-1 in SIF. Thereby, a residual DAO-1 activity of 8±0.1% was determined after an incubation of 90 min. In this food-matrix SIF, around 43% of the initially applied DAO-1 activity were available over 90 min. The results indicate that the stability of DAO-1 in SIF does not only depend on the amount of protein being added but also on the type of protein. BSA for example shows a higher stability in SIF than α-lactalbumin or β-lactoglobulin, which are subfractions of whey proteins. α-casein showed even weaker stability in SIF than the above-mentioned whey protein subfractions. β-lactoglobulin showed a high stability in a simulated gastric fluid due to its conformational state at the present pH that protects the target amino acids from pepsin digestion. At a higher pH, the conformational state of β-lactoglobulin makes it susceptible to the digestion of pancreatic enzymes like trypsin and chymotrypsin.

In conclusion, the stability of DAO-1 is difficult to assess under actual in vivo conditions due to the distinct influence of each compound present in a food matrix. However, being the most complex food-matrix and providing a sufficient DAO-1 stability, the food-matrix SIF 3 was used for all further experiments.

### Kinetics of DAO-1 in a Simulated Intestinal Fluid:

Besides the stability of DAO-1, also the kinetics in food-matrix containing SIF are of high importance for the histamine degradation capability. Therefore, kinetic investigations with DAO-1 were done in food-matrix SIF 3 without pancreatin (unhydrolyzed SIF) with histamine concentrations ranging from 1.56 to 50 mM (FIG. 18 A). The linearization of the Michaelis-Menten kinetics, according to Hanes-Woolf, is shown in FIG. 19. Furthermore, the kinetics were also investigated in Food-matrix SIF 3 with hydrolysis by pancreatin for 90 min at 37° C. prior to the kinetic investigations (FIG. 18 B) (hydrolyzed SIF). For DAO-1 a substrate inhibition was recognized for histamine concentrations greater than 12.5 mM for both the unhydrolyzed and hydrolyzed SIF food matrices. The Kₘ-value of DAO-1 for histamine under unhydrolyzed conditions was 5.7±0.5 mM (R²=0.99) and is thereby higher than the Kₘ-value of DAO-1 under optimal conditions in PIPES buffer (25 mM, pH 7.2) with a Kₘ-value of 2.3±+0.2 mM. In hydrolyzed SIF, DAO-1 showed a slightly lower Kₘ-value of 4.2±0.5 mM (R²=0.97) as compared to the unhydrolyzed SIF. Therefore, accumulation of free amino acids and peptides from the pancreatic digestion of the simulated food-matrix did not negatively affect the kinetics of DAO-1. The affinity of DAO-1 towards the substrate histamine is much lower when compared to DAO from human kidney with a Kₘ-value of 0.0028 mM. However, human DAO is not only relevant for the degradation of exogenous histamine in the small intestine but also seems to play an important role in the regulation of endogenous histamine in the blood plasma. A respective study considered a plasma histamine concentration of 100 ng/ml being a concentration that can induce a severe anaphylaxis reaction in humans. Therefore, human DAO must be able to degrade histamine at low concentrations with a high efficiency. The histamine concentration found in foods, especially in fermented foods, exceeds the concentration of histamine in human blood serum by far. For different types of cheese, concentrations between <0.1 mg/kg to 2500 mg/kg were found. For samples of red wine and dry-fermented sausages, maximum histamine concentrations of 55 mg/kg and 358 mg/kg, respectively, were reported. A high affinity of DAO to its substrates is advantageous as it results in higher conversion rates. However, under intestinal conditions, kinetics of DAO does not seem to have the same relevance as it does in blood serum since the histamine concentration present is by far higher.

### Tableting of DAO-1:

For the preparation of one DAO-1 tablet, the DAO-1 activity obtained from the disruption and purification of wet yeast cells from around 370 mL bioreactor volume was used (690 nkat) (Table 2). The SDS-PAGE analysis of this DAO-1 preparation showed a distinct band at around 75 kDa indicating the DAO-1 (FIG. 20). The purified DAO-1 was concentrated almost 90-fold by ammonium-sulfate precipitation, whereby no DAO-1 activity was lost. Before freeze drying, catalase from Micrococcus lysodeikticus was added to improve the stability and activity of DAO-1 under bioconversion conditions by removing accumulating hydrogen peroxide. It was observed in preliminary experiments that the used catalase withstands the freeze-drying process with no considerable enzyme activity loss. Freeze-drying of the purified DAO-1 in sodium phosphate buffer (20 mM, pH 7) with 40 g/L sucrose did not result in any DAO-1 activity losses. The DAO-1 containing powder was compressed in a self-built tablet press to a tablet length of 9 mm (7 mm diameter). The finished DAO-1 tablets showed sufficient stability and maintained their shape after extracting them from the tablet press.

TABLE 2
Details for the finished DAO-1 tablet.
DAO-1 tablet details (for 1 tablet)

| | |
|---|---|
| DAO-1 activity [nkat] | 690 |
| Catalase activity [μkat] | 14.9 |
| Protein content [mg] | 44 |
| Saccharose content [mg] | 238 |
| Total weight [mg] | 400 |
| Size [mm] | 9 × 7 (length × diameter) |

### Quantification of Histamine in Food-Matrix SIF Samples by RP-HPLC:

The quantitative determination of histamine in the food-matrix containing SIF is a difficult task due to the complex sample matrix. There, high saccharose and protein contents but more importantly the number of different peptides and free amino acids, generated from the proteolytic digestion, disturb the analysis of histamine by RP-HPLC. A derivatization of histamine with ortho-phthalaldehyde (OPA) would lead to the derivatization of various hydrolysis products and is thereby not applicable for this analytical problem. Therefore, the sample must be purified before the RP-HPLC analysis removing a majority of the foreign compounds.

Large molecules were removed from the crude sample by size exclusion with PD MidiTrap G-25 columns. Then, the histamine in this sample can be bound to a cation exchange material under acidic conditions due to its positive charge. Thereby, histamine can be separated from other substances by washing out all unbound compounds and eluting histamine with a pH-shift to alkaline conditions. The obtained histamine sample was separated on a RP-HPLC without any derivatization (FIG. 21). Histamine standards for the calibration were done in the respective SIF matrix and were treated as described above. The calibration showed sufficient linearity within a range of 0.1 to 2 mM histamine (R²=>0.994) (FIG. 22). The limit of detection and quantification was at 0.5 and 0.65 mM, respectively. The recovery of histamine standards (1.35 mM) was at 106.7% providing sufficient accuracy for the investigation of the histamine bioconversion using DAO-1 tablets.

### Histamine Bioconversion in a Simulated Intestinal Fluid Using DAO-1 Tablets:

The supplementation of porcine DAO to support the endogenous DAO in the small intestine has been evaluated in several clinical studies, which found the DAO supplementation to reduce histamine-associated physiological symptoms. In contrast to the findings in one study, it was recently shown in an in vitro study, that no DAO activity was detectable in the porcine DAO supplement and that at least 50 nkat of DAO activity are required for the degradation of food-relevant histamine amounts (75 mg). This histamine amount has been used in clinical studies to identify histamine intolerant humans. However, the required DAO activity was estimated only for the used buffer system and did not include the lowered DAO activity and kinetics under SIF conditions or the low stability due to the pancreatic digestion. Therefore, even higher DAO-1 activities might be required to obtain a satisfactory histamine reduction under SIF conditions.

The prepared DAO-1 tablet reduced the initially applied histamine concentration (1.35 mM; 150 mg/L; 75 mg) in the food-matrix SIF 3 by 29.3±0.8% in 90 min at 37° C. (FIG. 23). This equaled a total degradation of 22 mg histamine. The DAO-1 has previously been tested in a similar bioconversion experiment but under buffer conditions. Thereby, the histamine was reduced by 75%. A complete conversion of histamine was not possible due to the kinetics of DAO-1. To compensate for the loss of DAO-1 activity through proteolytic digestion, two tablets could be administered instead of one. This would most likely lead to a more efficient total histamine degradation. However, to obtain this required DAO-1 activity, the production should first be further improved investigating alternative expression hosts for the DAO-1 production. It is important to understand that the applied benchmark of 75 mg histamine is only a theoretical value and that the consumption of histamine containing meals can be accompanied with lower total histamine amounts. Furthermore, this amount even seems to be enough to provoke typical symptoms of a histamine intolerance in healthy individuals. Therefore, the achieved total degradation of 22 mg using one DAO-1 tablet might already be sufficient to support the endogenous DAO-mediated histamine degradation in the human small intestine. Further research including clinical studies must be done to assess the actual impact of DAO-1 tablets in histamine intolerant humans.

### Example 9

Recombinant Production of DAO-1 in Komagataella phaffii

The yeast Komagataella phaffii was genetically modified for the production of DAO-1 (from Y. lipolytica). For this purpose, the DAO-1 gene, codon-optimized for K. phaffii, was integrated into the GAP locus under the control of the GAP promoter. After integration, different clones were selected by an activity-based screening for the strongest DAO-1 expression capacity. The resulting K. phaffii_DAO-1 clone could then be used for the recombinant expression of DAO-1. The expression of DAO-1 under control of the constitutive and powerful GAP promoter can be done in a glucose-containing minimal medium. In addition, integration into the genome of K. phaffii can completely eliminate the need for antibiotics during production.

K. phaffii also has EFSA-issued (European Food Safety Authority) QPS (Qualified Presumption of Safety) status and FDA (Food and Drug Administration) GRAS (Generally Recognized As Safe) status and can thus be used for DAO-1 production with the aim of administration in humans.

K. phaffii_DAO-1 was cultured in a bioreactor in a glucose-containing minimal medium. Using an exponential feed (fed-batch cultivation) consisting of a glucose solution and trace elements, a maximum bio-wet and bio-dry mass of about 450 g/L and 115 g/L, respectively, could be achieved after a cultivation period of just under 36 h. A maximum DAO-1 activity of about 50,000 nkat/L_{medium} with a specific DAO-1 activity of 2500 nkat/gₚᵣₒₜₑᵢₙ could be achieved. Thus, compared to DAO-1 production in Y. lipolytica PO1f, not only could the cultivation time for achieving maximum DAO-1 activity be shortened by 20 h, but the activity yield could also be increased 18-fold.

DAO-1 productivity can be further increased in K. phaffii by using the methanol-inducible AOX1 promoter. Using a β-galactosidase reference enzyme, it could already be shown that the expression capacity could be increased 2-fold by using the AOX1 promoter compared to the GAP promoter. Accordingly, productivities of up to 100,000 nkat/L_{medium} DAO-1 can be expected.

### Discussion

Y. lipolytica PO1f was identified as the producer of a DAO (DAO-1) which showed a broad substrate selectivity, including the most relevant biogenic amines histamine, putrescine, cadaverine, and tyramine. This seems to be a rarely found feature in microbial DAOs, which makes DAO-1 an interesting enzyme for administration in the food industry. The DAO-1 showed similar biochemical characteristics regarding the temperature and pH profile compared to porcine and human DAOs. As observed for other microbial DAOs, DAO-1 showed a lower affinity (Kₘ=2.3±0.2 mM) towards the substrate histamine compared to the mammalian porcine and human DAOs. Nevertheless, DAO-1 was capable of reducing around 75% of the histamine applied (150 mg/L) in a histamine bioconversion experiment. The cost-effective and convenient production of DAO-1 in Y. lipolytica PO1f and its biochemical characteristics makes it an interesting enzyme for application in the food industry for the degradation of biogenic amines such as histamine, as well as for a range of medical applications in the prevention and treatment of conditions and diseases associated with the ingestion of biogenic amines.

Specifically, in the present invention, a putative diamine oxidase (DAO) from Yarrowia lipolytica PO1f (DAO-1) was homologously recombinantly integrated into the genome of Y. lipolytica PO1f using the CRISPR-Cas9 system for the subsequent DAO production in a bioreactor. Thereby, it was proven that the DAO-1 produced was indeed a functional DAO. The cultivation yielded 2343±98 nkat/L culture with a specific DAO activity of 1301±54.2 nkat/g_{Protein}, which was a 93-fold increase of specific DAO activity compared to the native Y. lipolytica PO1f DAO-1 production. The DAO-1 was most active at 40° C., pH 7.2 in Tris-HCl buffer (50 mM) (with histamine as substrate), which is comparable to human and porcine DAOs. With its broad selectivity for the most relevant biogenic amines in foods, DAO-1 from Y. lipolytica PO1f is an interesting enzyme for application in the food industry for the degradation of biogenic amines, as well as for a range of medical applications in the prevention and treatment of conditions and diseases associated with the ingestion of biogenic amines.

Further, DAO-1 from Y. lipolytica was investigated for its potential in the reduction of histamine under simulated intestinal conditions. Therefore, the purified DAO-1 was formulated as a sucrose-based tablet containing 690 nkat of DAO-1 activity. The tablet also contained a catalase from Micrococcus lysodeikticus ensuring that no accumulating hydrogen peroxide would inactivate the DAO-1 during the histamine degradation. It was shown for the first time using this DAO-1 tablet that actual food-relevant histamine amounts (22 mg) can be degraded with a microbial DAO under SIF conditions. This is an amount that could already be sufficient to circumvent symptoms of a histamine intolerance, supporting the endogenous histamine degradation.

The present invention relates to the following amino acid and nucleotide sequences:

Amino acid sequence of DAO-1 from Y. lipolytica PO1f
SEQ ID NO: 1
MTPHPFDQLSVQEMESVVRVVKSNHSGKSLHLKSIGTEEPPKALMAPFLAAKRAGKNPVPP
PRIAHVIYYVLEDKLVNQCWVDVPSAKVVKSEVLKKGIHPPIDPWEANEAFEAAFDHPLVK
DAIKKCGVEHLIDNLTIDGWMYGCDSEIDMPRYLQMLVYCRDPKTNHQDSNMYAFPVPFVP
VYDVLEKKLVRVDYCATGGDDDDAAVEGVGNYDTRPEGKNCIEHCVTNDYLPELQDKMRTD
LKPYNVLQPEGPSYHIDSDGYINWQKWHFKVGFTPREGLVIHDVHYDGRSTFYRLSMSEMA
VPYADPRPPLHRKMAFDFGDCGGGKCANELTLGCDCLGTIRYFDGNVCDPEGNVFTRKNVI
CMHEQDDGIGWKHTNYRTDVVAITRRRILVLQTILTVGNYEYIFAWHFDQSAGIQLEIRAT
GIVSTQLIDAGKKSKFGTIVSPGVMAASHQHIFNVRMDPAIDGHQNTVVVNDTVALPWDAK
NPHGIAFENTKTPIEKSCYLDSDIQKNRYLKICNENKINPISGNPVGYKIGGLATAMLYAQ
PGSVSRNRAAFATHHYWVTKYKDQELFAGGVWTNQSANEVGGVQDAVARNENVRNDDVVLW
HSFGLTHHPRVEDFPVMPCEIMKIHLSPNDFFTGNPSVDVPKSNQTFNRSVEVKDCRSCKI
Gene dao-1 from Y. lipolytica PO1f
SEQ ID NO: 2
atgactccccaccctttcgatcagctctccgttcaggagatggagagcgttgtgcgagtgg
tcaagtccaaccattcgggcaagtctcttcacctcaagtccattggcaccgaggagcctcc
caaggcgctgatggctcctttccttgcagccaagcgAgctggcaagaaccccgttcccccc
cctcgaatcgcacatgtcatctactatgttctggaggacaagttggtgaaccagtgctggg
tcgatgttccttccgccaaggttgtcaagtccgaggtgctcaagaagggcatccatcctcc
cattgatccctgggaggccaacgaggccttcgaggccgcctttgaccatcctctggtcaag
gacgccatcaagaagtgcggcgtggagcatctgatcgacaacctcacaattgacggctgga
tgtacggctgtgacagcgagattgacatgccccggtacctgcagatgctggtctactgccg
agatcccaagaccaaccaccaggactccaacatgtacgccttccccgttccgtttgttcct
gtctacgacgtgctggagaagaagctcgttcgagtcgactattgcgccaccggtggagacg
atgacgacgctgccgtcgagggcgttggcaactacgacacccgacccgagggaaagaactg
catcgagcactgtgtcaccaacgactatcttcccgagcttcaggacaagatgcgaaccgac
ctcaagccctacaacgtgttgcagcccgagggtccctcttaccacattgacagtgacggct
acatcaactggcaaaagtggcacttcaaggtcggattcactccccgagagggtctggtgat
ccacgatgtccactacgacggccgatccaccttctaccgactgtccatgtccgagatggcg
gttccctacgccgatccccggccccctctgcaccgaaagatggcgtttgatttcggcgact
gtggaggAggaaagtgcgccaacgagctgactctgggctgcgactgtcttggtaccatccg
atactttgacggcaacgtgtgcgaccccgagggcaacgtgttcacccgaaagaacgtcatc
tgtatgcacgagcaggatgacggtatcggctggaagcacactaactaccgaaccgacgtgg
ttgccatcacccgacgacgaattctggttctgcagaccattctgaccgtgggcaactacga
gtacatctttgcctggcactttgaccagtctgccggaatccagctggagatccgagccacc
ggaatcgtctccacccagcttatcgacgccggcaagaagtccaagtttggcaccattgtct
ctcccggagtcatggccgcctctcaccagcacattttcaacgtgcgaatggaccctgccat
tgacggccatcagaacacagttgtggtcaacgacaccgttgctctgccctgggacgccaag
aacccccatggaatcgcctttgagaacaccaagacccccatcgagaagtcgtgctacctgg
actcggacatccagaagaaccggtacctcaagatctgcaacgagaacaagatcaaccccat
ctccggcaaccccgtgggCtacaagattggaggtctggccaccgccatgctgtacgctcag
cccggctccgtttcgcgaaaccgagccgcctttgccacccaccactactgggtcaccaagt
acaaggaccaggagctgtttgccggCggcgtgtggaccaaccagtctgccaacgaggtcgg
cggagtccaggatgccgttgcccgaaacgagaacgtgcgaaacgacgacgtggttctctgg
cactcctttggtctcactcaccacccccgagtcgaggacttccccgtcatgccttgcgaga
ttatgaagatccatctgtcgcccaacgatttcttcaccggcaacccttctgttgacgtgcc
caagtccaaccagactttcaatcgatccgtcgaggtcaaggactgtcgatcttgcaagatc
tag

Nucleotides in underlined, bold capital letters indicate silent mutations with respect to the putative DNA sequence given in the NCBI database.

Gene dao-2 from Y. lipolytica PO1f
SEQ ID NO: 3
atgcacagactatcacaactagctacacaaacaaccgcggccaccatcaccgcaggtcatc
ctcttgatcctctctctccctccgaaatcgaacatgccgcttccatcgtcaaatcgcagat
gcgagactcgtctccgtaccggttcaatctcatcacgctgatcgagccgcccaaggccgag
cttcttgcgtgggaggcgtcgccttcctcggtggccaaacctcctcgacgagcggaggtcg
tactcgttgtgagaggcaagaagggcgtcaccgagggccgggtctgtctcaccggctcaaa
ggtgctttcgtggtccgaaattgagggcgtccagcctattcttaccgttgacgacctccaa
aaggtcgaggaaattgtgcgacaggaccccgaagtcatcaagcagtgcaaactcattggag
ttgacaacatgtcccaggtgtactgtgatccctggactattggctatgacgaaagatgggg
tgccgaacgacgtctacagcaggcgttcctctacttccgagcccaccaggacgactcccag
tactcccatcctcttgacttcactccaatctacgacgccacggagcagaaagtcatcttta
ttgatattcctcccgttcgtcgacctctctccaagcttaagaattccaacttcaaccctca
ggatatctccaagactaccggttacagagacgtgaagcccatcgacgtgtctcagcccgaa
ggagtcaacttcaagatgaccggtcgaatcatggagtggcagggattccgatttcacgtgg
gattcaactacagagagggaattgtgctgtctcagatctctttcaacgaccatggtaacca
gcgaaacatgttccatcgtctctctctcgttgaaatggttgttccctacggaaaccccgag
caccctcaccagcgaaagcacgcctttgatctgggagagtacggagccggtctcatgacca
atcctctttccctcggatgtgattgcaagggagtcattcactaccttgatgcgcacttttc
cgacgccgaaggaaagcctctcactgttcccaacgctgtgtgtatccacgaggaggacaat
ggtctgcttttcaagcactctgacttccgagacgagttccagacttcgatcgtcactcgag
ctaccaagctcattttgtcgcagattttcaccgccgcaaactacgaatactgcgtctactg
gattttccaccaggacggtaccatccagctggagattaagctcaccggcatcctcaacact
ttcccctgcaatcccggagaagatttgcatggctggggcacagaggtgtaccctggtacca
acgcccacaaccatcagcatctcttttctctgcgaatccatcctgccattgattcccagct
gcatccaggtaattctgtggcaatggtggacgctgagcggtcacccttcccgcctggacac
ccagaaaacctgcacggcaatggtttccggcccaagcgaacggtcttcaacaacccgatcg
aggctatgacggattatgatggtaatacatcgcgaacttgggactttttcaacccgaagtc
catcaaccagtactccaagaagcccgcttcttacaagctggtgtctcgtgagtgccctcct
ctgcttcctcagcctggtggactggtttggaaccgagctggttttgcacgacaccatatgc
atgttgttccgtatgtggacggccagctgtaccctgctggacggtttgtttgccagacaag
tggaaagccctcaaggggtctccccgagtggattgagcagtgcggagagaaggccaatatc
aacgataccgatgttgttgcctatcacacttttggtctgacccatttccctgctcccgagg
acttccctttgatgcctgccgagcccatgactctacttttgcgacccagaaacttcttcct
gcagaacccggctttggacgtgcctccttcgcatgctcgaaccaccacagaggcccaggct
gcttccggtgctaaggttgtctctcttaccgacaaggtgtcgcagctggctttcaataagt
cgtgctgttccaagtag
Forward primer for the amplification of dao-1 from the genomic DNA of Y. lipolytica
PO1f
SEQ ID NO: 4
atgactccccaccctttcgatcag
Reverse primer for the amplification of dao-1 from the genomic DNA of Y. lipolytica
PO1f
SEQ ID NO: 5
ctagatcttgcaagatcgacagtccttg
Forward primer for the second amplification of dao-1 and addition of restriction
sites
SEQ ID NO: 6
cttgcgcgcatgactccccaccctttc
Reverse primer for the second amplification of dao-1 and addition of restriction
sites
SEQ ID NO: 7
ggcgctagcctagatcttgcaagatcg
Forward primer for the amplification of dao-2 from the genomic DNA of Y. lipolytica
PO1f and addition of restriction sites
SEQ ID NO: 8
gaagcgcgcatgcacagactatcacaactagc
Reverse primer for the amplification of dao-2 from the genomic DNA of Y. lipolytica
PO1f and addition of restriction sites
SEQ ID NO: 9
caagctagcctacttggaacagcacga
Forward sequencing primer for dao genes
SEQ ID NO: 10
ctaaagatgttgatctccttgtgcc
Reverse sequencing primer for dao genes
SEQ ID NO: 11
cctctgggccgaatacaacac

1. A functional food or dietary supplement, comprising an enzyme having diamine oxidase activity, wherein said enzyme comprises
(i) the amino acid sequence of SEQ ID NO: 1; or
(ii) an amino acid sequence having at least 70% sequence identity to SEQ ID NO: 1 and having diamine oxidase activity.

2. (canceled)

3. A method for the production of a biogenic amine-depleted product, comprising the step of contacting (i) a biogenic amine-containing product, and/or (ii) a biogenic amine-containing intermediate product of said product, with an enzyme having diamine oxidase activity under conditions and for a duration of time suitable to degrade a biogenic amine present in said product and/or in said intermediate product of said product, wherein said enzyme comprises
(i) the amino acid sequence of SEQ ID NO: 1; or
(ii) an amino acid sequence having at least 70% sequence identity to SEQ ID NO: 1 and having diamine oxidase activity.

4. An enzyme having diamine oxidase activity for use in medicine, wherein said enzyme comprises
(i) the amino acid sequence of SEQ ID NO: 1; or
(ii) an amino acid sequence having at least 70% sequence identity to SEQ ID NO: 1 and having diamine oxidase activity.

5. The method according to claim 3, wherein the enzyme consists of
(i) the amino acid sequence of SEQ ID NO: 1; or
(ii) the amino acid sequence having at least 70% sequence identity to SEQ ID NO: 1 and having diamine oxidase activity.

6. The method according to claim 3, wherein the amino acid sequence in (ii) has at least 72%, at least 74%, at least 76%, at least 78%, at least 80%, at least 82%, at least 84%, at least 86%, at least 88%, at least 90%, at least 91%, at least 92%, at least 93%, at least 94%, at least 95%, at least 95.5%, at least 96%, at least 96.5%, at least 97%, at least 97.5%, at least 98%, at least 98.2%, at least 98.4%, at least 98.6%, at least 98.8%, at least 99%, at least 99.1%, at least 99.2%, at least 99.3%, at least 99.4%, at least 99.5%, at least 99.55%, at least 99.6%, at least 99.65%, at least 99.7%, at least 99.75%, at least 99.8%, at least 99.85%, at least 99.9%, at least 99.91%, at least 99.92%, at least 99.93%, at least 99.94%, at least 99.95%, at least 99.96%, at least 99.97%, at least 99.98%, or at least 99.9% sequence identity to SEQ ID NO: 1.

7. The method according to claim 3, wherein the enzyme consists of the amino acid sequence of SEQ ID NO: 1.

8. The method according to claim 3, wherein the product is selected from the group consisting of foodstuffs and feedstuffs.

9. The method according to claim 8, wherein the product is a foodstuff, selected from the group consisting of fermented foodstuffs, cheeses, sauerkraut, sausages, wine, chocolate, yeast extracts, fish, fish products, raw meats, vegetables, dairy products, and fresh milk.

10. A method for preventing or treating a condition or disease in a subject that is associated with increased levels of biogenic amines in a subject, comprising administering an enzyme to the subject an enzyme comprising
(i) the amino acid sequence of SEQ ID NO: 1; or
(ii) an amino acid sequence having at least 70% sequence identity to SEQ ID NO: 1 and having diamine oxidase activity.

11. The method according to claim 10, wherein the increased levels of biogenic amines are due to the ingestion of said biogenic amines.

12. The method according to claim 10, wherein the condition or disease that is associated with increased levels of biogenic amines is selected from the group consisting of allergies, acute and chronic allergic diseases, allergic reactions, allergy-like reactions, itching (pruritus), diarrhea, redness (erubescence), vomiting (emesis), acute and chronic biogenic amine poisoning, hypotonia, difficulty of breathing, biogenic amine intolerance, anaphylaxis, anaphylactic shock, acute and chronic urticaria, asthma, hay fever, allergic rhinitis, allergic conjunctivitis, headache, migraine, atopic dermatitis, mastocytosis, mast cell activation syndrome (MCAS), pre-eclampsia, hyperemesis gravidarum, pre-term labor, peptic ulcers, acid reflux, sepsis, fibromyalgia, chronic fatigue syndrome, and spondylitis.

13. The method according to claim 3, wherein the biogenic amine is selected from the group consisting of histamine, tyramine, putrescine, cadaverine, agmatine, spermidine, and tryptamine.

14. The method according to claim 13, wherein the biogenic amine is selected from the group consisting of histamine, tyramine, putrescine, and cadaverine.

15. The method according to claim 14, wherein the biogenic amine is histamine.
